(19) United States
(12) Patent Application Publication
(10) Pub. No.: US 2016/0194335 A1
(43) Pub. Date: Jul. 7, 2016

(54) POLYCYCLIC-CARBAMOYLPYRIDONE COMPOUNDS AND THEIR PHARMACEUTICAL USE

(71) Applicant: Gilead Sciences, Inc., Foster City, CA (US)

(72) Inventors: Haolun Jin, Foster City, CA (US); Scott E. Lazerwith, San Francisco, CA (US); Hyung-Jung Pyun, Fremont, CA (US)

(21) Appl. No.: 14/849,453

(22) Filed: Sep. 9, 2015

Related U.S. Application Data
(63) Continuation of application No. 14/629,290, filed on Feb. 23, 2015, now abandoned.
Application No. 14/629,290 is a continuation of application No. 14/133,855, filed on Dec. 19, 2013, now abandoned.
(60) Provisional application No. 61/788,397, filed on Mar. 15, 2013.
(60) Provisional application No. 61/745,375, filed on Dec. 21, 2012.

(51) Int. Cl. C07D 498/04; C07D 471/18; C07D 487/18

(52) U.S. Cl. CPC C07D 498/04; C07D 487/18; C07D 471/18

(57) ABSTRACT

Compounds for use in the treatment of human immunodeficiency virus (HIV) infection are disclosed. The compounds have the following Formula (I):

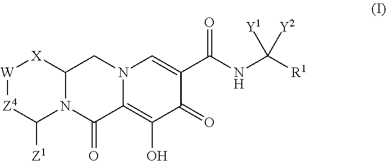

including stereoisomers and pharmaceutically acceptable salts thereof, wherein R¹, X, W, Y¹, Y², Z¹, Z², or Z⁴ are as defined herein. Methods associated with preparation and use of such compounds, as well as pharmaceutical compositions comprising such compounds, are also disclosed.

### CROSS-REFERENCE TO RELATED APPLICATIONS

This patent application is a Continuation of U.S. patent application Ser. No. 14/629,290, filed on Feb. 23, 2015, which is a continuation of U.S. application Ser. No. 14/133,855, filed on Dec. 19, 2013 which claims the benefit under 35 U.S.C. §119(e) of U.S. Provisional Patent Application No. 61/745,375, filed Dec. 21, 2012, and U.S. Provisional Patent Application No. 61/788,397, filed Mar. 15, 2013. The foregoing applications are incorporated herein by reference in their entireties.

### BACKGROUND

1. Field

Compounds, compositions, and methods for the treatment of human immunodeficiency virus (HIV) infection are disclosed. In particular, novel polycyclic carbamoylpyridone compounds and methods for their preparation and use as therapeutic or prophylactic agents are disclosed.

2. Description of the Related Art

Human immunodeficiency virus infection and related diseases are a major public health problem worldwide. Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, et al. N. Engl. J Med. (1998) 338:853-860; Richman, D. D. Nature (2001) 410:995-1001).

Pregnane X receptor (PXR) is a nuclear receptor that is one of the key regulators of enzymes involved in metabolism and elimination of small molecules from the body. Activation of PXR is known to up-regulate or induce the production of metabolic enzymes such as cytochrome P450 3A4 (CYP3A4) as well as enzymes involved in transport such as OATP2 in the liver and intestine (Endocrine Reviews (2002) 23(5):687-702). When one drug causes the up-regulation of these and other enzymes by activation of PXR, this can reduce the absorption and/or exposure of a co-administered drug that is susceptible to the up-regulated enzymes. To minimize the risk of this type of drug-drug interaction, it is desirable to minimize PXR activation. Further, it is known that PXR is activated by many different classes of molecules (Endocrine Reviews (2002) 23(5):687-702). Thus for drugs that will be co-administered with other drugs, it is important to test for and minimize PXR activation.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes. (Panel on Antiretroviral Guidelines for Adults and Adolescents. Guidelines for the use of antiretroviral agents in HIV-1-infected adults and adolescents. Department of Health and Human Services. Available at http://aidsinfo.nih.gov/ContentFiles/AdultandAdolescentGL.pdf. Section accessed Mar. 14, 2013.) In addition, decisions regarding the treatment of HIV infected patients is complicated when the patient requires treatment for other medical conditions (Id. at E-12). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

Accordingly, there is a need for new agents that inhibit the replication of HIV and that minimize PXR activation when co-administered with other drugs.

### BRIEF SUMMARY

The present invention is directed to novel polycyclic carbamoylpyridone compounds, having antiviral activity, including stereoisomers and pharmaceutically acceptable salts thereof, and the use of such compounds in the treatment of HIV infections. The compounds of the invention may be used to inhibit the activity of HIV integrase and may be used to reduce HIV replication.

In one embodiment of the present invention, compounds having the following Formula (I) are provided:

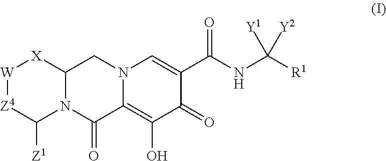

or a stereoisomer or pharmaceutically acceptable salt thereof,
wherein:

X is —O— or —NZ³— or —CHZ³—;

W is —O— or —NZ²— or —CHZ²—;

Z¹, Z² and Z³ are each, independently, hydrogen or C₁₋₃alkyl, or wherein Z¹ and Z² or Z¹ and Z³, taken together, form -L- wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—;

Z⁴ is a bond or —CH₂—, —CH₂CH₂—, —CH₂CH₂CH₂—, —CH₂OCH₂—, —CH₂NR^{a}CH₂—, —CH₂SCH₂—, —CH₂S(O)CH₂— or —CH₂SO₂CH₂—;

Y¹ and Y² are each, independently, hydrogen, C₁₋₃alkyl or C₁₋₃haloalkyl, or Y¹ and Y², together with the carbon atom to which they are attached, form a carbocyclic ring having from 3 to 6 ring atoms or a heterocyclic ring having from 3 to 6 ring atoms, wherein the carbocyclic or heterocyclic ring is optionally substituted with one or more R^{a};

R¹ is optionally substituted aryl or optionally substituted heteroaryl; and

each R^{a} is, independently, hydrogen, halo, hydroxyl or C₁₋₄alkyl, or wherein two R^{a} groups, together with the carbon atom to which they are attached, form ═O, and

wherein at least one of: (i) Z¹ and Z² or Z¹ and Z³, taken together, form -L-; or (ii) Y¹ and Y², together with the carbon atom to which they are attached, form a carbocyclic ring having from 3 to 5 ring atoms or a heterocyclic ring having from 3 to 5 ring atoms.

In another embodiment, a pharmaceutical composition is provided comprising a compound having Formula (I), or a stereoisomer or pharmaceutically acceptable salt thereof, and a pharmaceutically acceptable carrier, diluent or excipient.

The invention also provides the use of a pharmaceutical composition as described hereinabove for the treatment of an HIV infection in a human being having or at risk of having the infection.

In another embodiment, a method of using a compound having Formula (I) in therapy is provided. In particular, a method of treating the proliferation of the HIV virus, treating AIDS, or delaying the onset of AIDS or ARC symptoms in a mammal (e.g. a human) is provided, comprising administering to the mammal a compound having Formula (I), or a stereoisomer or pharmaceutically acceptable salt thereof, and a pharmaceutically acceptable carrier, diluent or excipient.

In another embodiment, use of a compound of Formula (I) as described herein, or a pharmaceutically acceptable salt thereof, for the treatment of an HIV infection in a human being having or at risk of having the infection is disclosed.

In another embodiment, the use of a compound of Formula (I) as described herein, or a pharmaceutically acceptable salt thereof, for the manufacture of a medicament for the treatment of an HIV infection in a human being having or at risk of having the infection is disclosed.

In another embodiment, an article of manufacture comprising a composition effective to treat an HIV infection; and packaging material comprising a label which indicates that the composition can be used to treat infection by HIV is disclosed. Exemplary compositions comprise a compound of Formula (I) according to this invention or a pharmaceutically acceptable salt thereof.

In still another embodiment, a method of inhibiting the replication of HIV is disclosed. The method comprises exposing the virus to an effective amount of the compound of Formula (I), or a salt thereof, under conditions where replication of HIV is inhibited.

In another embodiment, the use of a compound of Formula (I) to inhibit the activity of the HIV integrase enzyme is disclosed.

In another embodiment, the use of a compound of Formula (I), or a salt thereof, to inhibit the replication of HIV is disclosed.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DETAILED DESCRIPTION

In the following description, certain specific details are set forth in order to provide a thorough understanding of various embodiments of the invention. However, one skilled in the art will understand that the invention may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### DEFINITIONS

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Cyano” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, which is saturated or unsaturated (i.e., contains one or more double and/or triple bonds), having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —OR_{A} where R_{A} is an alkyl radical as defined above containing one to twelve carbon atoms. Unless stated otherwise specifically in the specification, an alkoxy group may be optionally substituted.

“Alkylamino” refers to a radical of the formula —NHR_{A} or —NR_{A}R_{A} where each R_{A} is, independently, an alkyl radical as defined above containing one to twelve carbon atoms. Unless stated otherwise specifically in the specification, an alkylamino group may be optionally substituted.

“Thioalkyl” refers to a radical of the formula —SR_{A} where R_{A} is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{B}—R_{C} where R_{B} is an alkylene chain as defined above and R_{C} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{B}R_{D} where R_{B} is an alkylene chain as defined above and R_{D} is a cycloalkyl radical as defined above. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, an N-heterocyclyl group may be optionally substituted.

“Heterocyclylalkyl” refers to a radical of the formula —R_{B}R_{E} where R_{B} is an alkylene chain as defined above and R_{E} is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; the nitrogen, carbon or sulfur atoms in the heteroaryl radical may be optionally oxidized; and the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{B}R_{F} where R_{B} is an alkylene chain as defined above and R_{F} is a heteroaryl radical as defined above. Unless stated otherwise specifically in the specification, a heteroarylalkyl group may be optionally substituted.

The term “substituted” used herein means any of the above groups (i.e., alkyl, alkylene, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{G}R_{H}, —NR_{G}C(═O)R_{H}, —NR_{G}C(═O)NR_{G}R_{H}, —NR_{G}C(═O)OR_{H}, —NR_{G}C(═NR_{g})NR_{G}R_{H}, —NR_{G}SO₂R_{H}, —OC(═O)NR_{G}R_{H}, —OR_{G}, —SR_{G}, —SOR_{G}, —SO₂R_{G}, —OSO₂R_{G}, —SO₂OR_{G}, ═NSO₂R_{G}, and —SO₂NR_{G}R_{H}. “Substituted also means any of the above groups in which one or more hydrogen atoms are replaced with —C(═O)R_{G}, —C(═O)OR_{G}, —C(═O)NR_{G}R_{H}, —CH₂SO₂R_{G}, —CH₂SO₂NR_{G}R_{H}. In the foregoing, R_{G} and R_{H} are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

The term “protecting group,” as used herein, refers to a labile chemical moiety which is known in the art to protect reactive groups including without limitation, hydroxyl and amino groups, against undesired reactions during synthetic procedures. Hydroxyl and amino groups protected with a protecting group are referred to herein as “protected hydroxyl groups” and “protected amino groups”, respectively. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999). Generally, groups are protected or present as a precursor that will be inert to reactions that modify other areas of the parent molecule for conversion into their final groups at an appropriate time. Further representative protecting or precursor groups are discussed in Agrawal, et al., Protocols for Oligonucleotide Conjugates, Eds, Humana Press; New Jersey, 1994; Vol. 26 pp. 1-72. Examples of “hydroxyl protecting groups” include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p-nitrobenzyl, triphenylmethyl, trimethylsilyl, triethylsilyl, t-butyldimethylsilyl, t-butyldiphenylsilyl (TBDPS), triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate. Examples of “amino protecting groups” include, but are not limited to, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide-protecting groups, such as 2-nitrobenzenesulfonyl; and imine and cyclic imide protecting groups, such as phthalimido and dithiasuccinoyl.

The invention disclosed herein is also meant to encompass all pharmaceutically acceptable compounds of Formula (I) being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵ and ¹³N, can be useful in Positron Emission Topography (PET) studies for examining substrate receptor occupancy. Isotopically-labeled compounds of Formula (I) can generally be prepared by conventional techniques known to those skilled in the art or by processes analogous to those described in the Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabeled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Examples of “pharmaceutically acceptable salts” of the compounds disclosed herein include salts derived from an appropriate base, such as an alkali metal (for example, sodium), an alkaline earth metal (for example, magnesium), ammonium and NX₄₊ (wherein X is C₁-C₄ alkyl). Pharmaceutically acceptable salts of a nitrogen atom or an amino group include for example salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, hydrobromic, sulfuric, phosphoric and sulfamic acids. Pharmaceutically acceptable salts of a compound of a hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds disclosed herein will typically be pharmaceutically acceptable, i.e. they will be salts derived from a physiologically acceptable acid or base. However, salts of acids or bases which are not pharmaceutically acceptable may also find use, for example, in the preparation or purification of a compound of formula I or another compound of the invention. All salts, whether or not derived from a physiologically acceptable acid or base, are within the scope of the present invention.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines. Finally, it is to be understood that the compositions herein comprise compounds disclosed herein in their un-ionized, as well as zwitterionic form, and combinations with stoichiometric amounts of water as in hydrates.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “treatment” as used herein is intended to mean the administration of a compound or composition according to the present invention to alleviate or eliminate symptoms of HIV infection and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood, and the administration of a compound or composition according to the present invention to prevent perinatal transmission of HIV from mother to baby, by administration to the mother before giving birth and to the child within the first days of life.

The term “antiviral agent” as used herein is intended to mean an agent (compound or biological) that is effective to inhibit the formation and/or replication of a virus in a human being, including but not limited to agents that interfere with either host or viral mechanisms necessary for the formation and/or replication of a virus in a human being.

The term “inhibitor of HIV replication” as used herein is intended to mean an agent capable of reducing or eliminating the ability of HIV to replicate in a host cell, whether in vitro, ex vivo or in vivo.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)-for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

### Compounds

As noted above, in one embodiment of the present invention, compounds having antiviral activity are provided, the compounds having the following Formula (I):

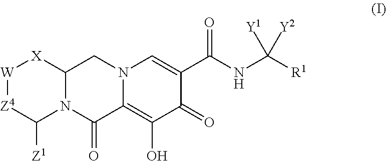

or a stereoisomer or pharmaceutically acceptable salt thereof,

wherein:

X is —O— or —NZ³— or —CHZ³—;

W is —O— or —NZ²— or —CHZ²—;

Z¹, Z² and Z³ are each, independently, hydrogen, C₁₋₃alkyl or C₁₋₃haloalkyl, or wherein Z¹ and Z² or Z¹ and Z³, taken together, form -L- wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—;

Z⁴ is a bond or —CH₂—, —CH₂CH₂—, —CH₂CH₂CH₂—, —CH₂OCH₂—, —CH₂NR^{a}CH₂—, —CH₂SCH₂—, —CH₂S(O)CH₂— or —CH₂SO₂CH₂—;

Y¹ and Y² are each, independently, hydrogen or C₁₋₃alkyl, or Y¹ and Y², together with the carbon atom to which they are attached, form a carbocyclic ring having from 3 to 6 ring atoms or a heterocyclic ring having from 3 to 6 ring atoms, wherein the carbocyclic or heterocyclic ring is optionally substituted with one or more R^{a};

R¹ is optionally substituted aryl or optionally substituted heteroaryl; and

each R^{a} is, independently, hydrogen, halo, hydroxyl or C₁₋₄alkyl, or wherein two R^{a} groups, together with the carbon atom to which they are attached, form ═O, and

wherein at least one of: (i) Z¹ and Z² or Z¹ and Z³, taken together, form -L-; or (ii) Y¹ and Y², together with the carbon atom to which they are attached, form a carbocyclic ring having from 3 to 5 ring atoms or a heterocyclic ring having from 3 to 5 ring atoms.

In another embodiment, Z¹ and Z² or Z and Z³, taken together, form -L-.

In another embodiment, compounds are provided having one of the following Formulas (II-A) or (II-B):

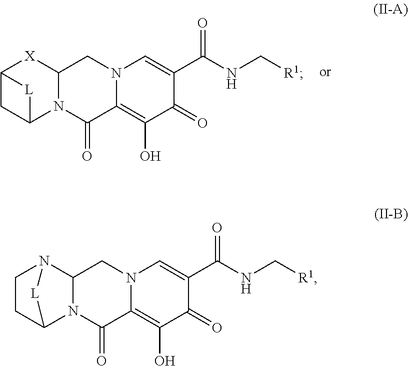

wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—.

In another embodiment, Y¹ and Y², together with the carbon atom to which they are attached, form a carbocyclic ring having from 3 to 5 ring atoms or a heterocyclic ring having from 3 to 5 ring atoms.

In another embodiment, compounds are provided having one of the following Formulas (III-A), (III-B), (III-C) or (III-D):

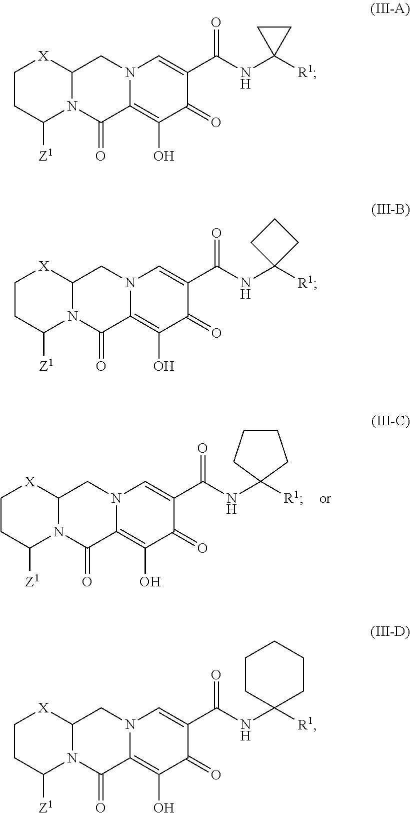

wherein Z¹ and Z³ are each, independently, hydrogen or C₁₋₃alkyl.

In another embodiment, compounds are provided having one of the following Formulas (III-E), (III-F), (III-G) or (III-H):

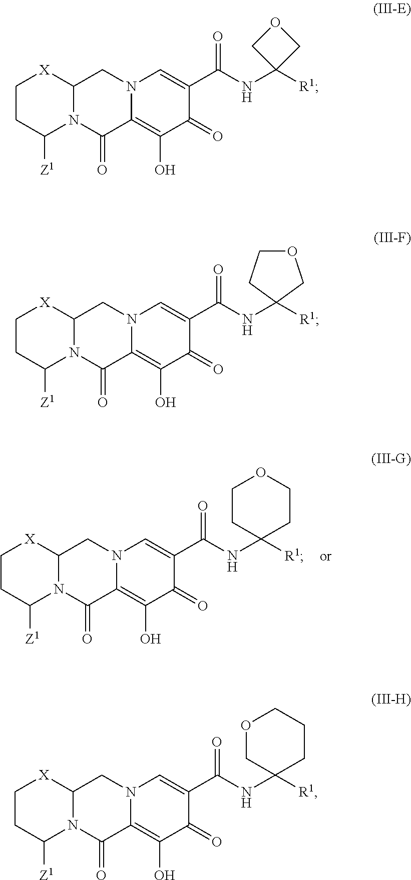

wherein Z¹ and Z³ are each, independently, hydrogen or C₁₋₃alkyl.

In another embodiment, both (i) Z¹ and Z² or Z¹ and Z³, taken together, form -L-, and (ii) Y¹ and Y², together with the carbon atom to which they are attached, form a carbocyclic ring having from 3 to 5 ring atoms or a heterocyclic ring having from 3 to 5 ring atoms.

In another embodiment, compounds are provided having one of the following Formulas (IV-AA), (IV-AB), (IV-AC), (IV-AD), (IV-AE), (IV-AF), (IV-AG) or (IV-AH):

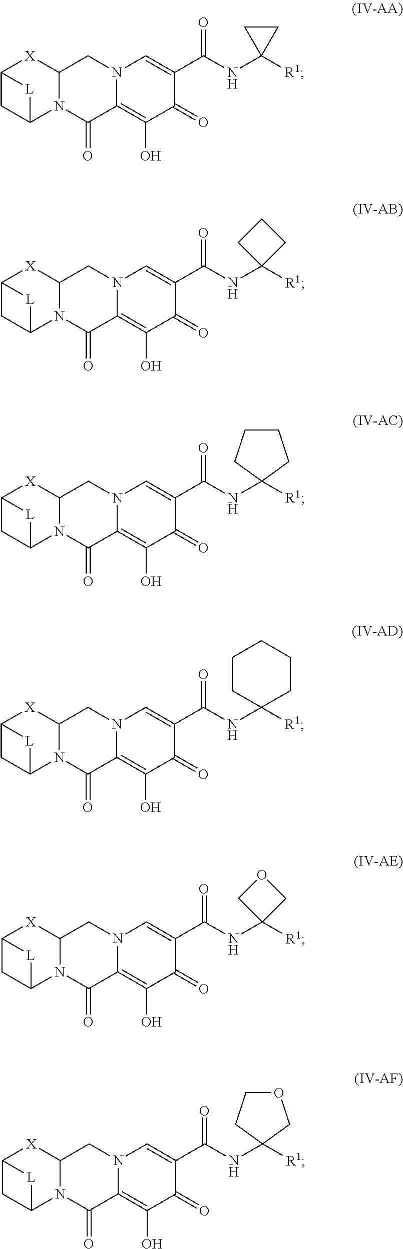

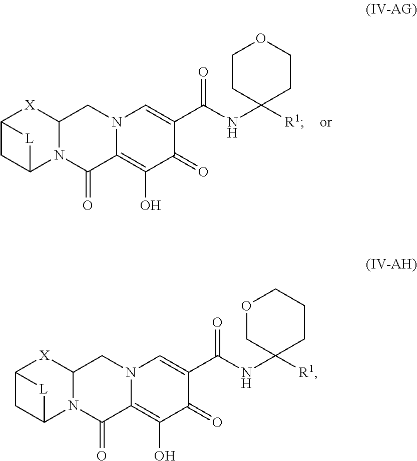

wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—.

In another embodiment, compounds are provided having one of the following Formulas (IV-BA), (IV-BB), (IV-BC), (IV-BD), (IV-BE), (IV-BF), (IV-BG) or (IV-BH):

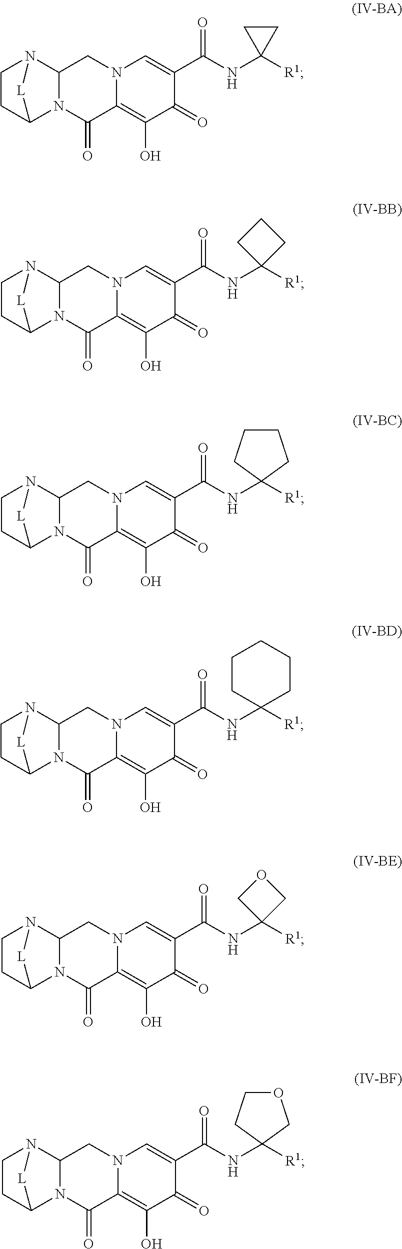

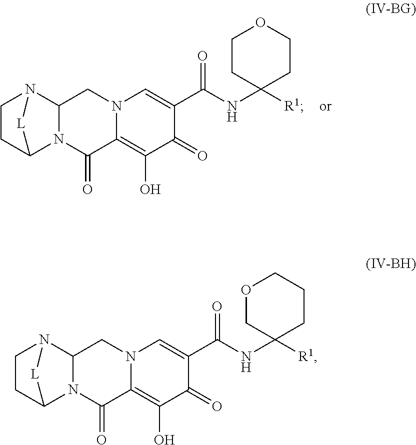

wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—.

In another embodiment, L is —C(R^{a})₂—, —C(R^{a})₂C(R^{a})₂—, —C(R^{a})₂C(R^{a})₂C(R^{a})₂—, or —C(R^{a})₂C(R^{a})₂C(R^{a})₂C(R^{a})₂—. In a further embodiment, L is —C(R^{a})₂—. In still a further embodiment, L is —C(R^{a})₂C(R^{a})₂—. In still a further embodiment, L is —C(R^{a})₂C(R^{a})₂C(R^{a})₂—. In still a further embodiment, each R^{a} is hydrogen.

In another embodiment, L is —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, or —C(R^{a})₂SO₂C(R^{a})₂—. In a further embodiment, each R^{a} is hydrogen.

In another embodiment, X is —O—. In another embodiment, X is —NH—. In another embodiment, X is —CH₂—.

In another embodiment, R¹ is aryl substituted with at least one halogen. In another embodiment, R¹ is aryl substituted with one or two halogens. In another embodiment, R¹ is 2,4-difluorophenyl, 4-fluorophenyl, 2,3-difluorophenyl, 3-fluoro-4-chlorophenyl, 3,4-difluorophenyl, 2-fluoro-4-chlorophenyl, 2-fluorophenyl, 3,5-difluorophenyl or 3-trifluoromethyl-4-fluorophenyl. For example, in another embodiment, R¹ is 2,4-difluorophenyl.

In one embodiment, a pharmaceutical composition is provided comprising a compound of any one of Formulas (I), (II-A), (II-B), (III-A), (III-B), (III-C), (III-D), (III-E), (III-F), (III-G), (III-H), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), or a stereoisomer or pharmaceutically acceptable salt thereof, and a pharmaceutically acceptable carrier, diluent or excipient.

Another embodiment is provided comprising a method of treating or preventing an HIV infection in a human having or at risk of having the infection by administering to the human a therapeutically effective amount of a compound of any one of Formulas (I), (II-A), (II-B), (III-A), (III-B), (III-C), (III-D), (III-E), (III-F), (III-G), (I-H), (I V-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), or a pharmaceutical composition thereof.

In another embodiment, the use of a compound of any one of Formulas (I), (II-A), (II-B), (III-A), (III-B), (III-C), (III-D), (III-E), (III-F), (III-G), (III-H), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), or a pharmaceutical composition thereof for the treatment or prevention of an HIV infection in a human having or at risk of having the infection.

It is understood that any embodiment of the compounds of Formulas (I), (II-A), (II-B), (III-A), (III-B), (III-C), (III-D), (III-E), (III-F), (III-G), (III-H), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), as set forth above, and any specific substituent set forth herein for a R¹, X, Y¹, Y², Z¹, Z², or Z⁴ group in the compounds of Formulas (I), (II-A), (II-B), (III-A), (III-B), (III-C), (III-D), (III-E), (III-F), (III-G), (III-H), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), as set forth above, may be independently combined with other embodiments and/or substituents of compounds of Formulas (I), (II-A), (II-B), (III-A), (III-B), (III-C), (III-D), (III-E), (III-F), (III-G), (III-H), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), to form embodiments of the inventions not specifically set forth above. In addition, in the event that a list of substitutents is listed for any particular L, R¹, R^{a}, X, Y¹, Y², Z¹, Z², Z³, or Z⁴ in a particular embodiment and/or claim, it is understood that each individual substituent may be deleted from the particular embodiment and/or claim and that the remaining list of substituents will be considered to be within the scope of the invention.

As one of skill in the art will appreciate, compounds of Formulas (I), (II-A), (II-B), (IV-AA), (IV-AB), (IV-AC), (IV-AD), (IV-AE), (IV-AF), (IV-AG), (IV-AH), (IV-BA), (IV-BB), (IV-BC), (IV-BD), (IV-BE), (IV-BF), (IV-BG), and (IV-BH), wherein Z¹ and Z² or Z¹ and Z³, taken together, form -L- may be shown in several different ways. For example, the Compound 3 of Example 3 may be shown as:

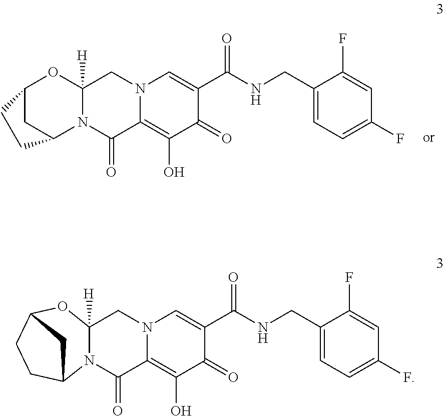 [SMILES: O=C(NCc1ccc(F)cc1F)c1cn2c(c(O)c1=O)C(=O)N1[C@@H]3CC[C@@H](C3)O[C@H]1C2 ; O=C(NCc1ccc(F)cc1F)c1cn2c(c(O)c1=O)C(=O)N1[C@@H]3CC[C@@H](C3)O[C@H]1C2]

### Pharmaceutical Compositions

For the purposes of administration, in certain embodiments, the compounds described herein are administered as a raw chemical or are formulated as pharmaceutical compositions. Pharmaceutical compositions of the present invention comprise a compound of Formula (I) and a pharmaceutically acceptable carrier, diluent or excipient. The compound of Formula (I) is present in the composition in an amount which is effective to treat a particular disease or condition of interest. The activity of compounds of Formula (I) can be determined by one skilled in the art, for example, as described in the Examples below. Appropriate concentrations and dosages can be readily determined by one skilled in the art.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

The following Examples illustrate various methods of making compounds of this invention, i.e., compound of Formula (I):

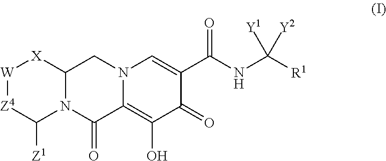

wherein R¹, X, W, Y¹, Y², Z¹, Z², or Z⁴ are as defined above. It is understood that one skilled in the art may be able to make these compounds by similar methods or by combining other methods known to one skilled in the art. It is also understood that one skilled in the art would be able to make, in a similar manner as described below, other compounds of Formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

The following examples are provided for purposes of illustration, not limitation.

### EXAMPLES

### General Synthetic Schemes

Schemes 1-3 are provided as further embodiments of the invention and illustrate general methods which were used to prepare compounds having Formula (I) and which can be used to prepare additional compound having Formula (I).

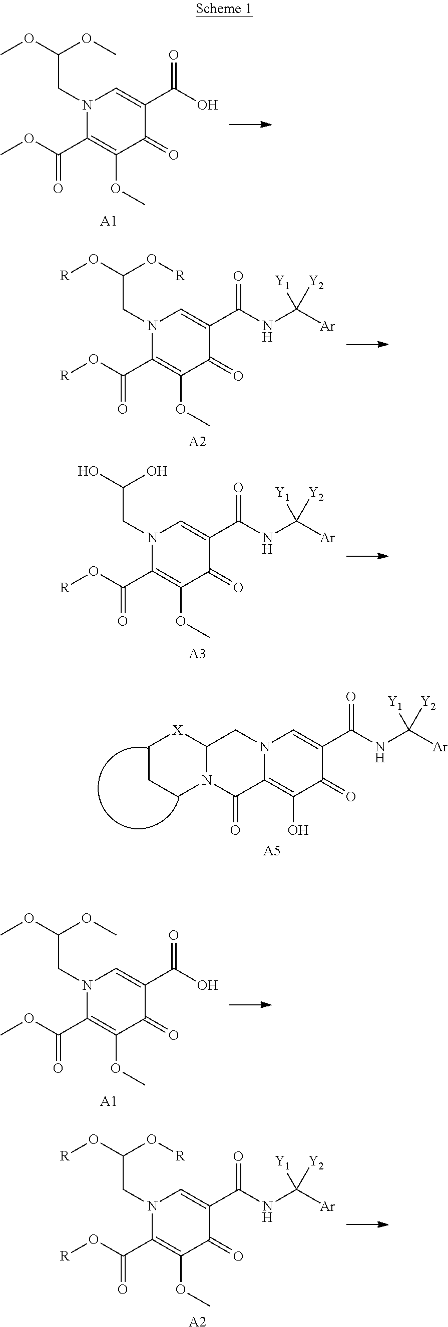

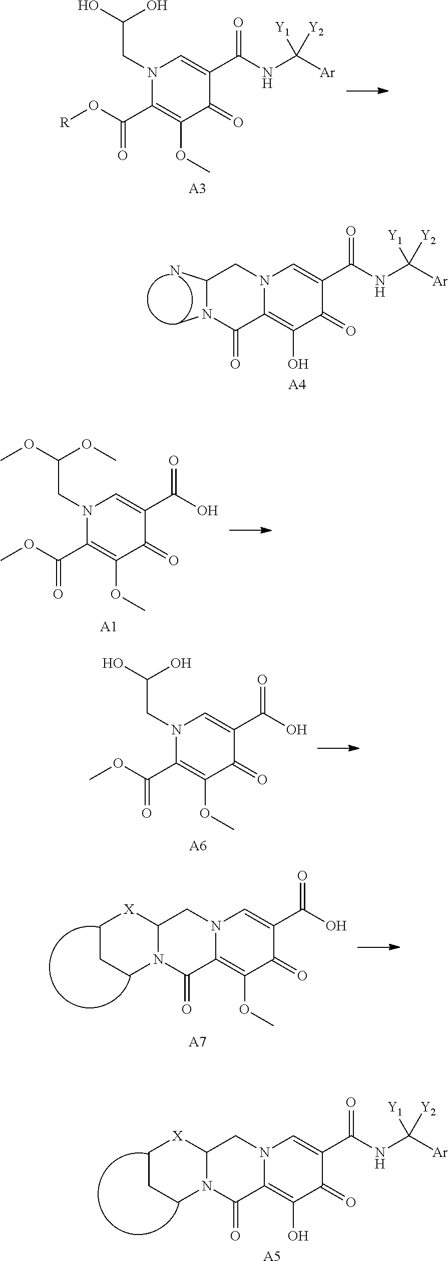

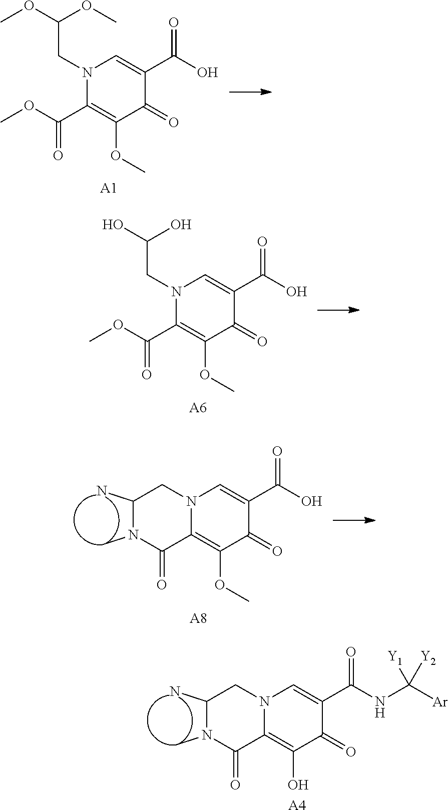

A1 can be converted to amide A2 with an appropriate amine and a coupling reagent such as HATU or EDCI. A2 can be converted to A3 with a strong acid such as methanesulfonic acid. A3 can be converted to either A5 or A4 by heating with an appropriate cyclic diamine or cyclic aminoalcohol followed by methyl deprotection with a reagent such as magnesium bromide.

Alternatively, A1 can be converted to A6 by treatment with a strong acid such as methanesulfonic acid. A6 can be condensed with an appropriate cyclic diamine or cyclic aminoalcohol followed by methyl deprotection with a reagent such as magnesium bromide to form either A7 or A8 respectively. A7 or A8 can be converted into amides A5 and A4 by treatment with an appropriate amine and a coupling reagent such as HATU or EDCI followed by methyl deprotection with a reagent such as magnesium bromide.

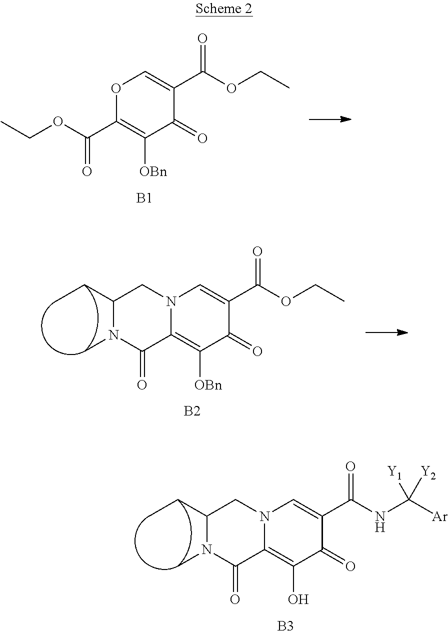

B1 (as described in WO2012/018065) is condensed with diamine under reflux condition to give B2. B2 is hydrolyzed and coupled with an amine by and amide-forming method to afford product B3 upon removal of a benzyl protecting group.

### Representative Compounds

### Example 1

### Preparation of Compound 1

### N-(2,4-difluorobenzyl)-8-hydroxy-7,9-dioxo-2,3,4,5,7,9,13, 13a-octahydro-2,5-methanopyrido[1′,2′:4,5]pyrazino[2,1-b][1,3]oxazepine-10-carboxamide

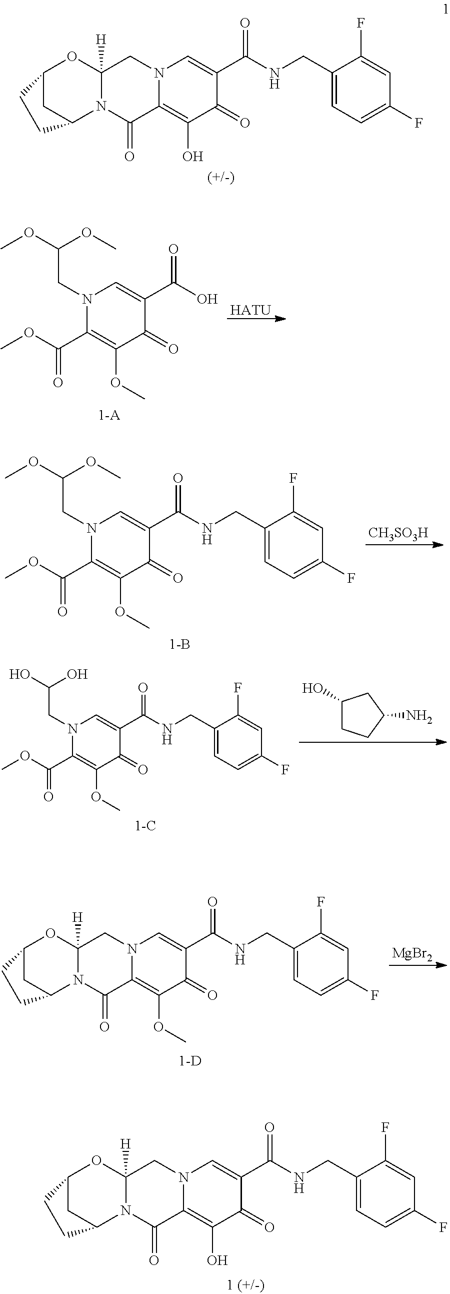

### Step 1

1-(2,2-dimethoxyethyl)-5-methoxy-6-(methoxycarbonyl)-4-oxo-1,4-dihydropyridine-3-carboxylic acid (1-A, 0.300 g, 0.95 mmol), prepared as described in WO2011/119566 A1, was evaporated once from dry toluene, suspended in acetonitrile (4 mL) and treated with diisopropylethylamine (0.329 mL, 1.90 mmol), 2,4-difluorobenzylamine (0.125 mL, 1.05 mmol) and HATU (0.433 g, 1.14 mmol). The reaction mixture was stirred for 10 minutes and concentrated. The residue was purified by flash chromatography on silica gel (10 to 60% ethyl acetate:dichloromethane) to afford the compound methyl 5-(2,4-difluorobenzylcarbamoyl)-1-(2,2-dimethoxyethyl)-3-methoxy-4-oxo-1,4-dihydropyridine-2-carboxylate, 1-B. ¹H-NMR (400 MHz, DMSO-d6) δ 10.28 (t, J=6.0 Hz, 1H), 8.46 (s, 1H), 7.42 (dd, J=15.4, 8.6 Hz, 1H), 7.24 (m, 1H), 7.06 (m, 1H), 4.52 (m, 3H), 4.22 (d, J=4.4 Hz, 2H), 3.92 (s, 3H), 3.80 (s, 3H), 3.29 (d, 6H). LCMS-ESI⁺ (m/z): [M+H]⁺ calculated for C₂₀H₂₃F₂N₂O₇: 441.15. found: 441.2.

### Step 2

Methyl 5-(2,4-difluorobenzylcarbamoyl)-1-(2,2-dimethoxyethyl)-3-methoxy-4-oxo-1,4-dihydropyridine-2-carboxylate (1-B, 0.106 g, 0.24 mmol) in acetonitrile (0.9 mL) and acetic acid (0.1 mL) was treated with methanesulfonic acid (0.005 mL, 0.072 mmol), sealed with a yellow cap, and heated to 70° C. After 16 hours, the mixture was cooled to afford a crude solution of methyl 5-(2,4-difluorobenzylcarbamoyl)-1-(2,2-dihydroxyethyl)-3-methoxy-4-oxo-1,4-dihydropyridine-2-carboxylate, 1-C. LCMS-ESI⁺ (m/z): [M+H]⁺ calculated for C₁₈H₁₉F₂N₂O₇: 413.12. found: 413.1.

### Steps 3 and 4

Methyl 5-(2,4-difluorobenzylcarbamoyl)-1-(2,2-dihydroxyethyl)-3-methoxy-4-oxo-1,4-dihydropyridine-2-carboxylate (1-C, 0.65 mL of the crude mixture from the previous step, 0.17 mmol) was treated with acetonitrile (0.65 mL) and cis-3-aminocyclpentanol (0.06 mL). The reaction mixture was sealed and heated to 90° C. After 30 minutes, the reaction mixture was cooled and magnesium bromide (0.063 g, 0.34 mmol) was added. The mixture was resealed and heated to 50° C. After 10 minutes, the reaction mixture was partitioned between dichloromethane and hydrochloric acid (0.2 M aq). The organic layer was removed and the aqueous layer extracted again with dichlormethane. The combined organic layers were dried over sodium sulfate, filtered and concentrated. Pre-HPLC purification (30-70% acetonitrile:water, 0.1% TFA) afforded Compound 1 as a racemic mixture. ¹H-NMR (400 MHz, DMSO-d6) δ 12.45 (br s, 1H), 10.35 (t, J=5.8 Hz, 1H), 8.45 (s, 1H), 7.37 (dd, J=15.4, 8.6 Hz, 1H), 7.23 (dt, J=2.5, 9.9 Hz, 1H), 7.05 (dt, J=2.2, 8.7 Hz, 1H), 5.43 (dd, J=9.6, 4.0 Hz, 1H), 5.09 (br s, 1H), 4.68 (dd, J=13.2, 4.0 Hz, 1H), 4.59 (br s, 1H), 4.53 (m, 2H), 4.02 (dd, J=12.6, 9.4 Hz), 1.93 (br s, 4H), 1.83 (d, J=12.0 Hz), 1.57 (dt, J=12.2, 3.2 Hz). LCMS-ESI⁺ (m/z): [M+H]⁺ calculated for C₂₁H₂₀F₂N₃O₅: 432.14. found: 432.2.

### Examples 2 and 3

### Preparation of Compounds 2 and 3

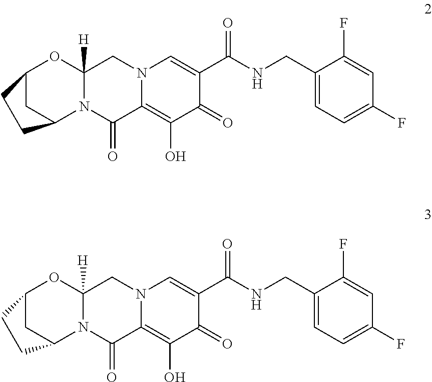 [SMILES: O=C(NCc1ccc(F)cc1F)c1cn2c(c(O)c1=O)C(=O)N1[C@H]3CC[C@H](C3)O[C@@H]1C2 ; O=C(NCc1ccc(F)cc1F)c1cn2c(c(O)c1=O)C(=O)N1[C@@H]3CC[C@@H](C3)O[C@H]1C2]

Compound 1 (16 mg) was separated by chiral HPLC using Chiralpak AS-H with 100% ethanol as eluent to afford Compounds 2 and 3 in enantiomerically enriched form. For Compound 2: LCMS-ESI⁺ (m/z): [M+H]¹ calculated for C₂₁H₂₀F₂N₃O₅: 432.14. found: 432.2, Chiral HPLC retention time=4.50 minutes (Chiralpak AS-H, 150×4.6 mm, 1 mL/min EtOH). For Compound 3: LCMS-ESI (m/z): [M+H]¹ calculated for C₂₁H₂₀F₂N₃O₅: 432.14. found: 432.2, Chiral HPLC retention time=6.84 minutes (Chiralpak AS-H, 150×4.6 mm, 1 mL/min EtOH). ¹H-NMR (400 MHz, DMSO-d6) δ 12.45 (br s, 1H), 10.35 (t, J=5.8 Hz, 1H), 8.44 (s, 1H), 7.37 (dd, J=15.2, 8.4 Hz, 1H), 7.23 (m, 1H), 7.05 (dt, J=1.8 Hz, 8.7 Hz, 1H), 5.44 (dd, J=9.6, 4.0 Hz), 5.09 (br s, 1H), 4.68 (dd, J=12.8, 4.0 Hz, 1H), 4.59 (br s, 1H), 4.53 (m, 2H), 4.02 (dd, J=12.6, 9.4 Hz, 1H), 1.93 (br s, 4H), 1.83 (d, J=12.4 Hz, 1H), 1.57 (m, 1H).

Alternatively, Compound 3 was prepared as follows:

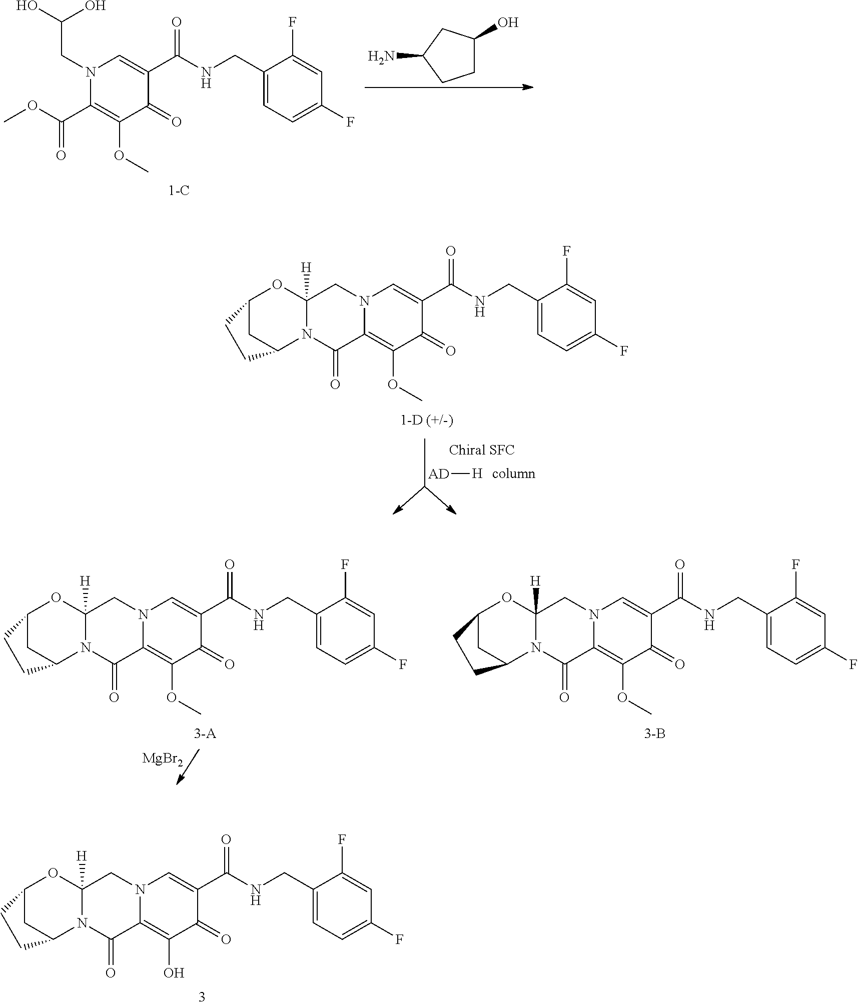

Methyl 5-(2,4-difluorobenzylcarbamoyl)-1-(2,2-dihydroxyethyl)-3-methoxy-4-oxo-1,4-dihydropyridine-2-carboxylate (1-C, 1.2 mmol in 5 mL of 9:1 acetonitrile:acetic acid containing 0.026 mL methanesulfonic acid) was treated with acetonitrile (5.0 mL) and cis-3-aminocyclpentanol (0.24 g, 2.4 mmol). The reaction mixture was sealed and heated to 90° C. After 30 minutes, the reaction mixture was cooled, treated with potassium carbonate (0.332 g, 2.4 mmol), sealed and reheated to 90° C. After 15 minutes, the mixture was cooled and partitioned between dichlormethane and hydrochloric acid (0.2 M aqueous). The organic layer was removed and the aqueous solution was extracted again with dichloromethane. The combined organic layers were dried over sodium sulfate (anhydrous), filtered and concentrated. The residue was purified by flash chromatography (0-8% ethanol (containing 11% saturated aqueous ammonium hydroxide) in dichloromethane) to afford Intermediate 1-D. LCMS-ESI⁺ (m/z): [M+H]⁺ calculated for C₂₂H₂₂F₂N₃O₅:446.15. found: 446.2.

Intermediate 1-D (270 mg) was separated by chiral SFC on a 50 mm Chiralpak AD-H column using 50% (1:1 methanol:acetonitrile) in supercritical carbon dioxide as eluent to afford Intermediates 3-A (first eluting peak) and 3-B (second eluting peak) in enantioenriched form. For 3-A: LCMS-ESI⁺ (m/z): [M+H]⁺ calculated for C₂₂H₂₂F₂N₃O₅:446.15. found: 446.2. For 3-B: LCMS-ESI⁺ (m/z): [M+H]⁺ calculated for C₂₂H₂₂F₂N₃O₅:446.15. found: 446.2.

Intermediate 3-A (0.110 g, 0.247 mmol) in acetonitrile (5 mL) was treated portion wise with magnesium bromide (0.091 g, 0.494 mmol), sealed and heated to 50° C. After 10 minutes the mixture was cooled and partitioned between dichloromethane and hydrochloric acid (0.2 M aqueous). The organic layer was separated and the aqueous extracted again with dichloromethane. The combined organic layers were dried over sodium sulfate, filtered and concentrated. Preparative HPLC purification (30-70% acetonitrile:water, 0.1% TFA) afforded Compound 3 in enantioenriched form. Chiral HPLC retention time=6.51 minutes (Chiralpak AS-H, 150×4.6 mm, 1 mL/min EtOH). LCMS-ESI⁺ (m/z): [M+H]⁺ calculated for C₂₁H₂₀F₂N₃O₅:432.14. found: 432.2. ¹H-NMR (400 MHz, DMSO-d6) δ 12.45 (br s, 1H), 10.35 (t, J=5.8 Hz, 1H), 8.44 (s, 1H), 7.37 (dd, J=15.2, 8.4 Hz, 1H), 7.23 (m, 1H), 7.05 (dt, J=1.8 Hz, 8.7 Hz, 1H), 5.44 (dd, J=9.6, 4.0 Hz), 5.09 (br s, 1H), 4.68 (dd, J=12.8, 4.0 Hz, 1H), 4.59 (br s, 1H), 4.53 (m, 2H), 4.02 (dd, J=12.6, 9.4 Hz, 1H), 1.93 (br s, 4H), 1.83 (d, J=12.4 Hz, 1H), 1.57 (m, 1H).

### Example 4

### Preparation of Compound 4

### (1S,4R)—N-(2,4-difluorobenzyl)-7-hydroxy-6,8-dioxo-3,4,6,8,12, 12a-hexahydro-2H-1,4-methanopyrido[1′,2′:4,5]pyrazino[1,2-a]pyrimidine-9-carboxamide

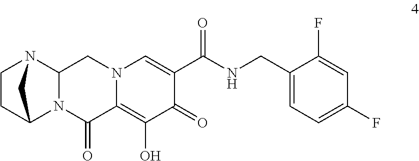 [SMILES: O=C(NCc1ccc(F)cc1F)c1cn2c(c(O)c1=O)C(=O)N1C(C2)[N@@]2CC[C@@H]1C2]

Methyl 5-(2,4-difluorobenzylcarbamoyl)-1-(2,2-dihydroxyethyl)-3-methoxy-4-oxo-1,4-dihydropyridine-2-carboxylate (1-C, 0.12 mmol in 0.53 mL of 9:1 acetonitrile:acetic acid containing 0.002 mL methanesulfonic acid) was treated with acetonitrile then (R)-pyrrolidin-3-amine (0.032 mL, 0.36 mmol). The reaction mixture was capped and heated to 90° C. for 5.5 hours. After cooling, the mixture was partitioned between dichloromethane and sodium bicarbonate (1M aqueous). The organic layer was separated and the aqueous was extracted again with ethyl acetate. The combined organic layers were dried over sodium sulfate (anhydrous), filtered and concentrated. The residue was dissolved in acetonitrile (1 mL), treated with magnesium bromide (0.022 g, 0.12 mmol), capped and heated to 50° C. for 10 minutes. After cooling the mixture was partitioned between dichloromethane and ammonium chloride (sat). The organic layer was separated and the aqueous was extracted again with dichloromethane. The aqueous layer was adjusted to pH=1 with HCl (aq) and extracted again with dichloromethane. The aqueous solution was adjusted to pH=3 with NaOH (aq) and extracted again with dichloromethane. The combined organic layers were dried over sodium sulfate, filtered, and concentrated. Preparative HPLC purification (10-55% acetonitrile:water, 0.1% TFA) afforded Compound 4. ¹H-NMR (400 MHz, CD₃OD-d4) δ 8.42 (s, 1H), 7.42, (q, J=7.7 Hz, 1H), 6.99-6.90 (m, 2H), 5.07 (br s, 1H), 4.73 (br d, J=10.8 Hz, 1H), 4.62 (s, 2H), 4.51 (br d, J=12.8 Hz, 1H), 4.07 (t, J=11.8 Hz, 1H), 3.4-3.0 (m, 3H), 2.76 (br d, J=8.8 Hz, 1H), 2.15-2.0 (m, 1H), 1.9-1.8 (m, 1H). LCMS-ESI⁺ (m/z): [M+H]⁺ calculated for C₂₀H₁₉F₂N₄O₄: 417.14. found: 417.2.

### Example 5

### Preparation of Compound 5

### (4R,12aS)—N-(1-(2,4-difluorophenyl)cyclopropyl)-7-hydroxy-4-methyl-6,8-dioxo-3,4,6,8,12,12a-hexahydro-2H-[1,3]oxazino[3,2-d]pyrido[1,2-a]pyrazine-9-carboxamide

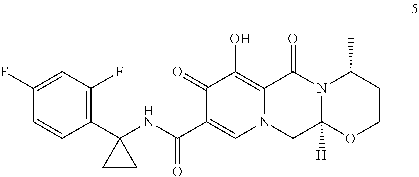 [SMILES: C[C@@H]1CCO[C@H]2Cn3cc(C(=O)NC4(c5ccc(F)cc5F)CC4)c(=O)c(O)c3C(=O)N12]

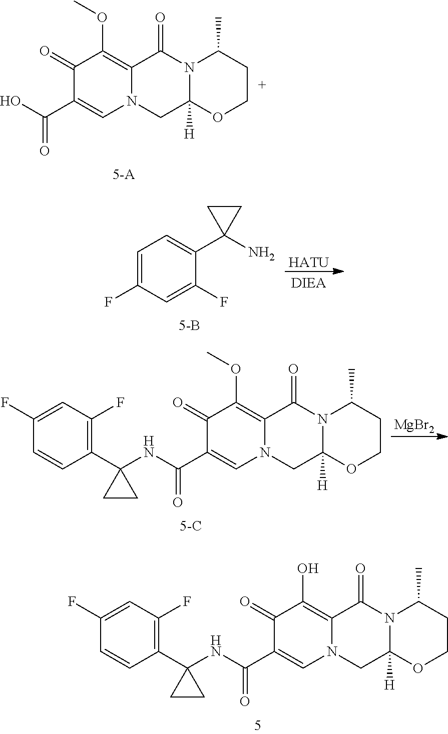

### Step 1

(4R,12aS)-7-methoxy-4-methyl-6,8-dioxo-3,4,6,8,12,12a-hexahydro-2H-[1,3]oxazino[3,2-d]pyrido[1,2-a]pyrazine-9-carboxylic acid (Intermediate 5-A) was prepared in an analogous manner to (3S,11aR)-6-methoxy-3-methyl-5,7-dioxo-2,3,5,7,11,11a-hexahydrooxazolo[3,2-d]pyrido[1,2-a]pyrazine-8-carboxylic acid as described in WO2011/119566, substituting (R)-3-aminobutan-1-ol for (S)-2-aminopropan-1-ol. WO2011/119566 is incorporated herein by reference in its entirety. A suspension of Intermediate 5-A (24.8 mg, 0.080 mmol), 1-(2,4-difluorophenyl)cyclopropanamine HCl salt (5-B, 21.9 mg, 0.107 mmol), and HATU (48 mg, 0.126 mmol) in CH₂Cl₂ (2 mL) was stirred at ambient temperature as diisopropylethylamine (0.1 mL, 0.574 mmol) was added. After 30 minutes, the reaction mixture was diluted with ethyl acetate before washing with 10% aqueous citric acid solution (×1) and saturated aqueous NaHCO₃ solution (×1). After the aqueous fractions were extracted with ethyl acetate (×1), the organic fractions were combined, dried (MgSO₄), and concentrated. The residue was purified by combiflash (12 g column) using hexanes, ethyl acetate, and 20% methanol in ethyl acetate to obtain (4R,12aS)—N-(1-(2,4-difluorophenyl)cyclopropyl)-7-methoxy-4-methyl-6,8-dioxo-3,4,6,8,12,12a-hexahydro-2H-[1,3]oxazino[3,2-d]pyrido[1,2-a]pyrazine-9-carboxamide, Intermediate 5-C. LCMS-ESI⁺ (m/z): [M+H]⁺ calculated for C₂₃H₂₄F₂N₃O₅:460.17. found 460.2.

### Step 2

A suspension of Intermediate 5-C (39 mg, 0.080 mmol) and magnesium bromide (42 mg, 0.2282 mmol) in acetonitrile (2 mL) was stirred at 50° C. After 1 hour, the reaction mixture was stirred at 0° C. bath when 1 N HCl (2 mL) was added. After the resulting mixture was diluted with water (˜20 mL), the product was extracted with dichloromethane (×3) and the combined extracts were dried (MgSO₄) and concentrated. The residue was purified by preparative HPLC to obtain (4R,12aS)—N-(1-(2,4-difluorophenyl)cyclopropyl)-7-hydroxy-4-methyl-6,8-dioxo-3,4,6,8,12,12a-hexahydro-2H-[1,3]oxazino[3,2-d]pyrido[1,2-a]pyrazine-9-carboxamide, compound 5, as TFA salt. ¹H-NMR (400 MHz, CDCl₃) δ 10.72 (br s, 1H), 8.37 (s, 1H), 7.57 (d, J=7.9 Hz, 1H), 6.71-6.81 (m, 2H), 5.23 (dd, J=5.6 and 4.4 Hz, 1H), 4.98 (br quint, J=˜6.5 Hz, 1H), 4.26 (dd, J=13.6 and 4.4 Hz, 1H), 4.12 (dd, J=13.6 and 5.6 Hz, 1H), 4.00-4.06 (m, 2H), 2.16-2.25 (m, 1H), 1.55 (br dd, J=13.8 and 1.8 Hz, 1H), 1.40 (d, J=6.8 Hz, 3H), 1.22-1.31 (m, 4H). ¹⁹F NMR (376.1 MHz, CDCl₃) δ −76.38 (s, 3F), −111.69 ˜−111.645 (m, 2F). LCMS-ESI⁺ (m/z): [M+H]⁺ calculated for C₂₂H₂₂F₂N₃O₅:446.15. found: 446.2.

### Example 6

### Preparation of Compound 6

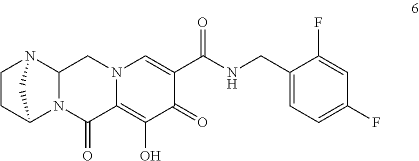 [SMILES: O=C(NCc1ccc(F)cc1F)c1cn2c(c(O)c1=O)C(=O)N1C(C2)[N@]2CC[C@H]1C2]

Methyl 5-(2,4-difluorobenzylcarbamoyl)-1-(2,2-dihydroxyethyl)-3-methoxy-4-oxo-1,4-dihydropyridine-2-carboxylate (1-C, 0.100 g, 0.243 mmol), (S)-pyrrolidin-3-amine (0.043 mL, 0.485 mmol) and potassium carbonate (0.067 g, 0.485 mmol) were suspended in acetonitrile (1.9 mL) and acetic acid (0.1 mL) and heated to 90° C. for 1.5 h. After cooling, the mixture was treated with magnesium bromide (0.090 g) and heated to 50° C. for 30 min. After cooling, the mixture partitioned between dichloromethane and 0.2 M HCl. The organic layer was separated and the aqueous was extracted again with dichloromethane. The combined organic layers were dried over sodium sulfate (anhydrous), filtered and concentrated. Preparative HPLC purification (25-50% acetonitrile:water, 0.1% TFA) afforded Compound 6. ¹H-NMR (400 MHz, DMSO-d₆) δ 10.33 (t, J=6.0 Hz, 1H), 8.44 (s, 1H), 7.48-7.32 (m, 1H), 7.31-7.15 (m, 1H), 7.14-6.97 (m, 1H), 4.86 (d, J=2.9 Hz, 1H), 4.62-4.54 (m, 1H), 4.52 (d, J=5.9 Hz, 1H), 4.01 (d, J=13.0 Hz, 1H), 2.99-2.76 (m, 3H), 1.96-1.81 (m, 1H), 1.71-1.53 (m, 1H). LCMS-ESI⁺ (m/z): [M+H]⁺ calculated for C₂₀H₁₉F₂N₄O₄: 417.14. found: 417.2.

### Example 7

### Preparation of Compound 7

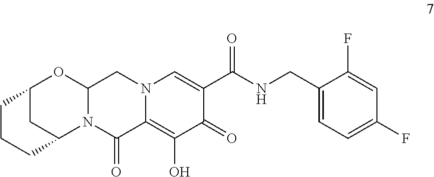 [SMILES: O=C(NCc1ccc(F)cc1F)c1cn2c(c(O)c1=O)C(=O)N1C(C2)O[C@H]2CCC[C@@H]1C2]

Methyl 5-(2,4-difluorobenzylcarbamoyl)-1-(2,2-dihydroxyethyl)-3-methoxy-4-oxo-1,4-dihydropyridine-2-carboxylate (1-C, 0.050 g, 0.121 mmol), (1S,3R)-3-aminocyclohexanol (0.028 g, 0.243 mmol) and potassium carbonate (0.034 g, 0.243 mmol) were suspended in acetonitrile (0.95 mL) and heated to 90° C. for 0.5 h. After cooling, acetoic acid (0.050 mL) was added and the mixture was reheated to 90° C. for 2 h. After cooling the mixture was treated with magnesium bromide (0.044 g) and heated to 50° C. for 1 h. After cooling, a second portion of magnesium bromide (0.044 g) was added and the mixture was reheated to 50° C. for 15 min. After cooling, the mixture partitioned between dichloromethane and 0.2 M HCl. The organic layer was separated and the aqueous was extracted again with dichloromethane. The combined organic layers were dried over sodium sulfate (anhydrous), filtered and concentrated. Preparative HPLC purification (40-80% acetonitrile:water, 0.1% TFA) afforded Compound 7. ¹H-NMR (400 MHz, DMSO-d₆) δ 12.40 (s, 1H), 10.36 (t, J=6.1 Hz, 1H), 8.45 (s, 1H), 7.48-7.29 (m, 1H), 7.31-7.13 (m, 1H), 7.13-6.97 (m, 1H), 5.56 (dd, J=10.0, 4.1 Hz, 1H), 4.70 (dd, J=12.7, 4.1 Hz, 1H), 4.52 (d, J=5.5 Hz, 2H), 4.40-4.29 (m, 2H), 4.06 (dd, J=12.5, 10.2 Hz, 1H), 2.46-2.36 (m, 1H), 1.98-1.63 (m, 4H), 1.57-1.30 (m, 3H). LCMS-ESI⁺ (m/z): [M+H]⁺ calculated for C₂₂H₂₂F₂N₃O₅: 446.15. found: 446.2.

### Example 8

### Preparation of Compound 8

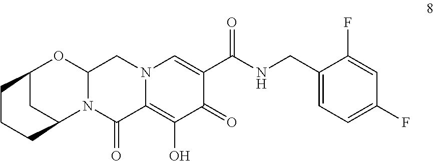 [SMILES: O=C(NCc1ccc(F)cc1F)c1cn2c(c(O)c1=O)C(=O)N1C(C2)O[C@@H]2CCC[C@H]1C2]

Compound 8 was prepared in a similar manner to compound 7 using (1R,3S)-3-aminocyclohexanol in place of (1S,3R)-3-aminocyclohexanol. ¹H-NMR (400 MHz, DMSO-d₆) δ 12.40 (s, 1H), 10.36 (t, J=6.1 Hz, 1H), 8.45 (s, 1H), 7.48-7.30 (m, 1H), 7.23 (td, J=10.6, 2.7 Hz, 1H), 7.05 (td, J=8.3, 2.3 Hz, 1H), 5.56 (dd, J=10.1, 4.1 Hz, 1H), 4.70 (dd, J=12.8, 3.9 Hz, 1H), 4.52 (d, J=5.6 Hz, 2H), 4.39-4.27 (m, 2H), 4.06 (dd, J=12.6, 10.0 Hz, 1H), 2.47-2.35 (m, 1H), 2.00-1.64 (m, 4H), 1.58-1.30 (m, 3H). LCMS-ESI⁺ (m/z): [M+H]⁺ calculated for C₂₂H₂₂F₂N₃O₅: 446.15. found: 446.2.

### Examples 9 and 10

### Preparation of Compounds 9 and 10

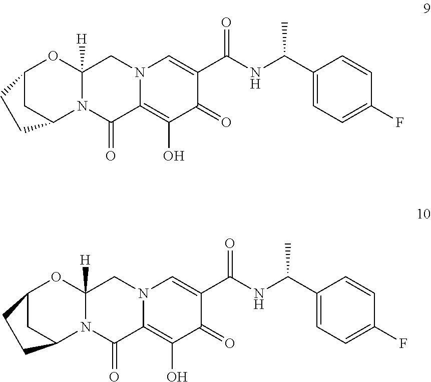 [SMILES: C[C@@H](NC(=O)c1cn2c(c(O)c1=O)C(=O)N1[C@@H]3CC[C@@H](C3)O[C@H]1C2)c1ccc(F)cc1 ; C[C@@H](NC(=O)c1cn2c(c(O)c1=O)C(=O)N1[C@H]3CC[C@H](C3)O[C@@H]1C2)c1ccc(F)cc1]

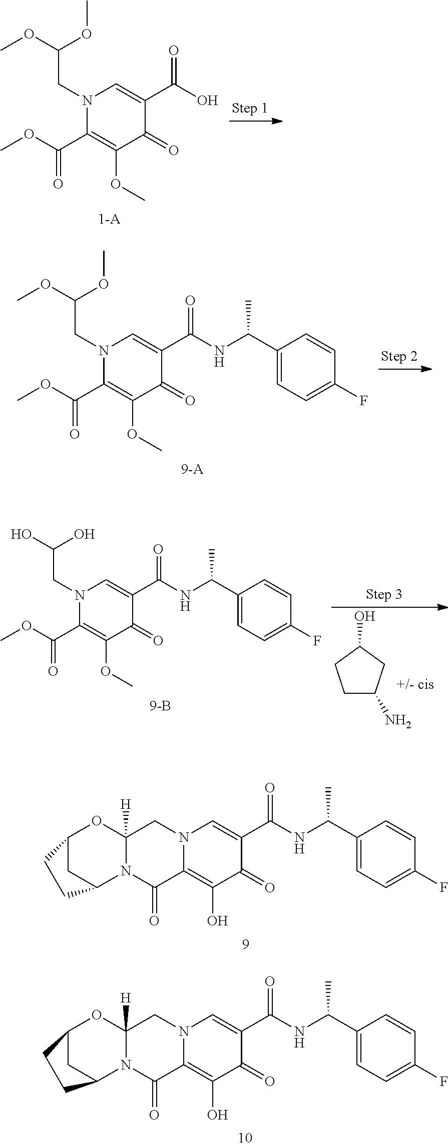

### Step 1

1-(2,2-dimethoxyethyl)-5-methoxy-6-(methoxycarbonyl)-4-oxo-1,4-dihydropyridine-3-carboxylic acid (1-A, 0.500 g, 1.59 mmol), was suspended in acetonitrile (6 mL) and treated with diisopropylethylamine (0.550 mL, 3.17 mmol), (R)-1-(4-fluorophenyl)ethanamine (0.242 mg, 1.74 mmol) and HATU (0.661 g, 1.74 mmol). The reaction mixture was stirred for 2 h and partitioned between ethyl acetate and water. The organic layer was separated and washed with HCl (10% aq), sodium bicarbonate (1M aq), dried over sodium sulfate, filtered and concentrated to afford crude (R)-methyl 1-(2,2-dimethoxyethyl)-5-(1-(4-fluorophenyl)ethylcarbamoyl)-3-methoxy-4-oxo-1,4-dihydropyridine-2-carboxylate which was used without purification in the next step: LCMS-ESI⁺ (m/z): [M+H]⁺ calculated for C₂₁H₂₆FN₂O₇: 437.17. found: 437.1.

### Step 2

(R)-methyl 1-(2,2-dimethoxyethyl)-5-(1-(4-fluorophenyl)ethylcarbamoyl)-3-methoxy-4-oxo-1,4-dihydropyridine-2-carboxylate was suspended in acetonitrile (5.7 mL) and acetic acid (0.6 mL) and treated with methane sulfonic acid (0.031 mL, 0.477 mmol). The mixture was capped and heated to 75° C. After 7 h, the mixture was cooled and used without purification in the next step: LCMS-ESI⁺ (m/z): [M+H]⁺ calculated for C₁₉H₂₂FN₂0₇: 409.14. found: 409.0.

### Step 3

(R)-methyl 1-(2,2-dihydroxyethyl)-5-(1-(4-fluorophenyl)ethylcarbamoyl)-3-methoxy-4-oxo-1,4-dihydropyridine-2-carboxylate (3.6 mL of the crude mixture from Step 2, 0.8 mmol) was diluted with acetonitrile (3.6 mL) and treated with cis-3-aminocyclpentanol, HCl salt (0.219 g, 1.6 mmol) and potassium carbonate (0.276 g, 2.0 mmol). The mixture was capped and heated to 90° C. After 20 min, the reaction mixture was cooled and partitioned between dichloromethane and HCl (0.2 M aq). The layers were separated and the aqueous layer was extracted again with dichloromethane. The combined organic layers were treated with a small amount of acetonitrile, dried over sodium sulfate, filtered and concentrated.

The residue was suspended in acetonitrile (4 mL) and treated with magnesium bromide (0.177 g). The mixture was capped and heated to 50° C. After 10 min, the reaction mixture was cooled and partitioned between dichloromethane and HCl (0.2 M aq). The layers were separated and the aqueous layer was extracted again with dichlormethane. The combined organic layers were dried over sodium sulfate, filtered and concentrated. The residue was purified by flash chromatography on silica gel (0-8% ethanol:DCM) to afford a diastereomeric mixture of desired 9 and 10.

The mixture was separated by chiral HPLC using Chiralpak AD-H with 100% ethanol as eluent to afford Compounds 9 and 10 in enantiomerically enriched form:

For Compound 9: LCMS-ESI⁺ (m/z): [M+H]⁺ calculated for C₂₂H₂₃FN₃O₅:428.16. found: 428.1. Chiral HPLC retention time=10.177 minutes (Chiralpak AD-H, 150×4.6 mm, 1 mL/min EtOH). ¹H-NMR (400 MHz, DMSO-d₆) δ 12.45 (s, 1H), 10.45 (d, J=7.7 Hz, 1H), 8.40 (s, 1H), 7.37 (dd, J=8.6, 5.6 Hz, 2H), 7.15 (t, J=8.9 Hz, 2H), 5.44 (dd, J=9.5, 4.2 Hz, 1H), 5.17-5.04 (m, 2H), 4.73-4.62 (m, 1H), 4.59 (s, 1H), 4.00 (dd, J=12.7, 9.5 Hz, 1H), 1.93 (s, 4H), 1.83 (d, J=11.8 Hz, 1H), 1.56 (dt, J=12.1, 3.4 Hz, 1H), 1.44 (d, J=6.9 Hz, 3H).

For Compound 10: LCMS-ESI⁺ (m/z): [M+H]⁺ calculated for C₂₂H₂₃FN₃O₅:428.16. found: 428.1. Chiral HPLC retention time=14.061 minutes (Chiralpak AD-H, 150×4.6 mm, 1 mL/min EtOH). ¹H-NMR (400 MHz, DMSO-d₆) δ 12.44 (s, 1H), 10.46 (d, J=7.8 Hz, 1H), 8.41 (s, 1H), 7.37 (dd, J=8.6, 5.6 Hz, 2H), 7.15 (t, J=8.9 Hz, 2H), 5.42 (dd, J=9.6, 4.1 Hz, 1H), 5.18-5.02 (m, 2H), 4.67 (dd, J=12.8, 4.2 Hz, 1H), 4.59 (s, 1H), 4.02 (dd, J=12.7, 9.6 Hz, 1H), 1.93 (s, 4H), 1.83 (d, J=12.0 Hz, 1H), 1.57 (dt, J=13.0, 3.5 Hz, 1H), 1.44 (d, J=6.9 Hz, 3H).

### Example 11

### Preparation of Compound 11

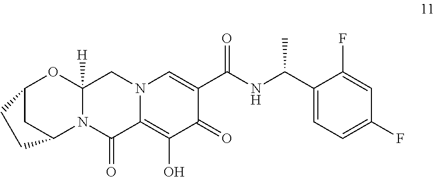 [SMILES: C[C@@H](NC(=O)c1cn2c(c(O)c1=O)C(=O)N1[C@@H]3CC[C@@H](C3)O[C@H]1C2)c1ccc(F)cc1F]

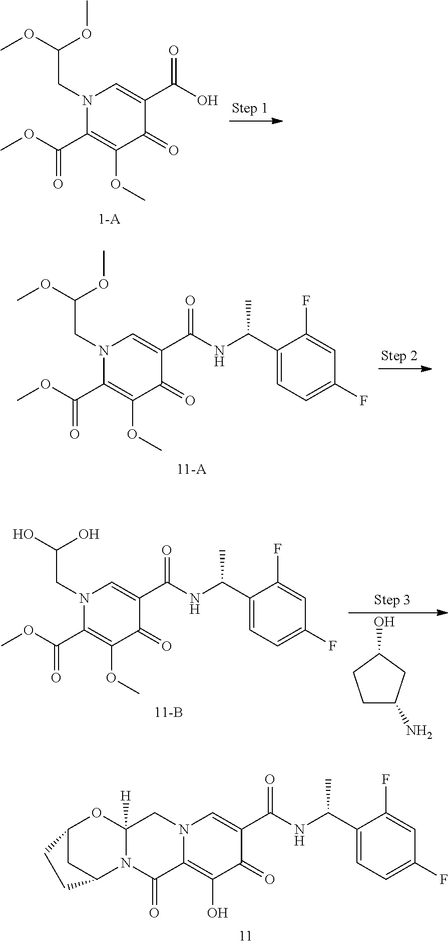

### Step 1

1-(2,2-dimethoxyethyl)-5-methoxy-6-(methoxycarbonyl)-4-oxo-1,4-dihydropyridine-3-carboxylic acid (1-A, 0.315 g, 1.00 mmol), was suspended in acetonitrile (4 mL) and treated with diisopropylethylamine (0.348 mL, 2.00 mmol), (R)-1-(2,4-difluorophenyl)ethanamine HCl salt (0.213 mg, 1.10 mmol) and HATU (0.418 g, 1.10 mmol). The reaction mixture was stirred for 1 h and partitioned between dichloromethane and HCl (10% aq). The organic layer was separated and washed sodium bicarbonate (1M aq), dried over sodium sulfate, filtered and concentrated to afford crude (R)-methyl 5-(1-(2,4-difluorophenyl)ethylcarbamoyl)-1-(2,2-dimethoxyethyl)-3-methoxy-4-oxo-1,4-dihydropyridine-2-carboxylate which was used without purification in the next step. LCMS-ESI⁺ (m/z): [M+H]⁺ calculated for C₂₁H₂₅F₂N₂O₇: 455.16. found: 455.1.

### Step 2

(R)-methyl 5-(1-(2,4-difluorophenyl)ethylcarbamoyl)-1-(2,2-dimethoxyethyl)-3-methoxy-4-oxo-1,4-dihydropyridine-2-carboxylate was suspended in acetonitrile (3.6 mL) and acetic acid (0.4 mL) and treated with methane sulfonic acid (0.020 mL). The mixture was capped and heated to 75° C. After 16 h, the crude mixture was cooled and used without purification in the next step. LCMS-ESI⁺ (m/z): [M+H]⁺ calculated for C₁₉H₂₁F₂N₂O₇: 427.13. found: 427.1.

### Step 3

(R)-methyl 5-(1-(2,4-difluorophenyl)ethylcarbamoyl)-1-(2,2-dihydroxyethyl)-3-methoxy-4-oxo-1,4-dihydropyridine-2-carboxylate (half of the crude mixture from Step 2, approx 0.5 mmol) was diluted with acetonitrile (2.5 mL) and treated with (1S,3R)-3-aminocyclopentanol (0.110 g, 1.09 mmol) and potassium carbonate (0.069 g, 0.50 mmol). The mixture was capped and heated to 90° C. After 15 min, the reaction mixture was cooled and magnesium bromide (0.184 g) was added. The reaction mixture was heated to 50° C. After 10 min, the mixture was cooled and treated with an additional portion of magnesium bromide (0.184 g). The reaction mixture was reheated to 50° C. and stirred for 10 min. After cooling, the mixture was partitioned between dichloromethane and HCl (0.2 M aq). The layers were separated and the aqueous layer was extracted again with dichloromethane. The combined organic layers were dried over sodium sulfate, filtered and concentrated. Preparative HPLC purification (30-60% acetonitrile:water, 0.1% TFA) afforded desired Compound 11. LCMS-ESI⁺ (m/z): [M+H]⁺ calculated for C₂₂H₂₂F₂N₃O₅:446.15. found: 446.1. ¹H-NMR (400 MHz, DMSO-d₆) δ 12.46 (s, 1H), 10.53 (d, J=7.5 Hz, 1H), 8.38 (s, 1H), 7.39 (q, J=8.5 Hz, 1H), 7.29-7.12 (m, 1H), 7.13-6.93 (m, 1H), 5.44 (dd, J=9.8, 4.2 Hz, 1H), 5.28 (p, J=7.3, 6.8 Hz, 1H), 5.09 (s, 1H), 4.66 (dd, J=13.2, 4.3 Hz, 1H), 4.59 (s, 1H), 3.99 (dd, J=13.1, 9.6 Hz, 1H), 1.93 (s, 4H), 1.83 (d, J=12.4 Hz, 1H), 1.56 (dt, J=12.5, 2.9 Hz, 1H), 1.45 (d, J=6.9 Hz, 3H).

### Example 12

### Preparation of Compound 12

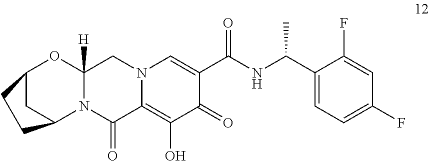 [SMILES: C[C@@H](NC(=O)c1cn2c(c(O)c1=O)C(=O)N1[C@H]3CC[C@H](C3)O[C@@H]1C2)c1ccc(F)cc1F]

Compound 12 was prepared in a similar manner to compound 11 using (1R,3S)-3-aminocyclopentanol in place of (1S,3R)-3-aminocyclopentanol. ¹H-NMR (400 MHz, DMSO-d₆) δ 12.43 (s, 1H), 10.52 (d, J=8.2 Hz, 1H), 8.38 (s, 1H), 7.39 (q, J=8.4 Hz, 1H), 7.28-7.12 (m, 1H), 7.11-6.97 (m, 1H), 5.41 (dd, J=10.0, 4.0 Hz, 1H), 5.35-5.20 (m, 1H), 5.08 (s, 1H), 4.65 (dd, J=13.1, 3.8 Hz, 1H), 4.58 (s, 1H), 4.01 (dd, J=12.8, 9.5 Hz, 1H), 1.92 (s, 4H), 1.83 (d, J=11.5 Hz, 1H), 1.61-1.51 (m, 1H), 1.44 (d, J=6.9 Hz, 3H). LCMS-ESI⁺ (m/z): [M+H]⁺ calculated for C₂₂H₂₂F₂N₃O₅:446.15. found: 446.1.

### Example 13

### Preparation of Compound 13

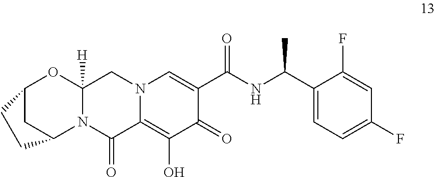 [SMILES: C[C@H](NC(=O)c1cn2c(c(O)c1=O)C(=O)N1[C@@H]3CC[C@@H](C3)O[C@H]1C2)c1ccc(F)cc1F]

Compound 13 was prepared in a similar manner to compound 11 using (S)-1-(2,4-difluorophenyl)ethanaminein place of (R)-1-(2,4-difluorophenyl)ethanamine, and using only a single portion of magnesium bromide (0.184 g). ¹H-NMR (400 MHz, DMSO-d₆) δ 12.44 (s, 1H), 10.53 (d, J=7.8 Hz, 1H), 8.39 (s, 1H), 7.39 (q, J=8.5 Hz, 1H), 7.32-7.14 (m, 1H), 7.05 (t, J=9.1 Hz, 1H), 5.42 (dd, J=9.5, 4.2 Hz, 1H), 5.29 (p, J=6.9 Hz, 1H), 5.09 (s, 1H), 4.65 (dd, J=12.9, 4.3 Hz, 1H), 4.59 (s, 1H), 4.02 (dd, J=12.6, 9.8 Hz, 1H), 1.92 (s, 4H), 1.83 (d, J=12.1 Hz, 1H), 1.61-1.52 (m, 1H), 1.44 (d, J=6.9 Hz, 3H). LCMS-ESI⁺ (m/z): [M+H]⁺ calculated for C₂₂H₂₂F₂N₃O₅:446.15. found: 446.2.

### Example 14

### Preparation of Compound 14

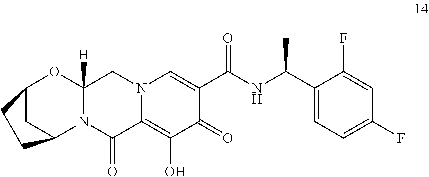 [SMILES: C[C@H](NC(=O)c1cn2c(c(O)c1=O)C(=O)N1[C@H]3CC[C@H](C3)O[C@@H]1C2)c1ccc(F)cc1F]

Compound 14 was prepared in a similar manner to compound 11 using (S)-1-(2,4-difluorophenyl)ethanamine in place of (R)-1-(2,4-difluorophenyl)ethanamine and using (1R,3S)-3-aminocyclopentanol in place of (1S,3R)-3-aminocyclopentanol: ¹H-NMR (400 MHz, DMSO-d₆) δ 12.46 (s, 1H), 10.53 (d, J=7.6 Hz, 1H), 8.38 (s, 1H), 7.39 (q, J=8.6 Hz, 1H), 7.28-7.14 (m, 1H), 7.05 (t, J=8.5 Hz, 1H), 5.44 (dd, J=9.8, 3.8 Hz, 1H), 5.28 (p, J=8.0 Hz, 1H), 5.09 (s, 1H), 4.66 (dd, J=12.9, 4.0 Hz, 1H), 4.59 (s, 1H), 3.99 (dd, J=12.5, 9.6 Hz, 1H), 1.93 (s, 4H), 1.83 (d, J=12.6 Hz, 1H), 1.56 (dt, J=13.0, 3.3 Hz, 1H), 1.45 (d, J=6.9 Hz, 3H). LCMS-ESI⁺ (m/z): [M+H]⁺ calculated for C₂₂H₂₂F₂N₃O₅:446.15. found: 446.1.

### Example 15

### Preparation of Compound 15

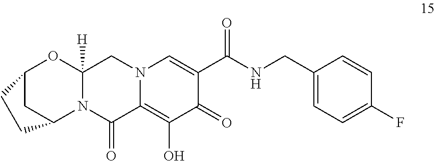 [SMILES: O=C(NCc1ccc(F)cc1)c1cn2c(c(O)c1=O)C(=O)N1[C@@H]3CC[C@@H](C3)O[C@H]1C2]

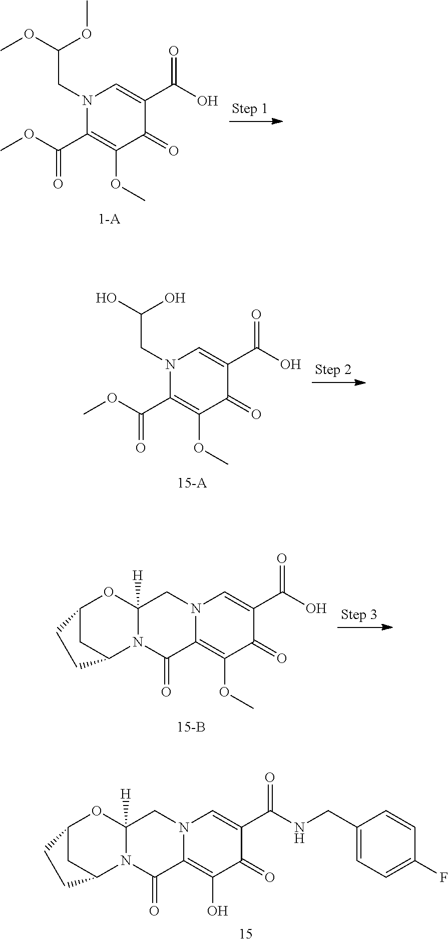 [SMILES: COC(=O)c1c(OC)c(=O)c(C(=O)O)cn1CC(OC)OC ; COC(=O)c1c(OC)c(=O)c(C(=O)O)cn1CC(O)O ; COc1c2n(cc(C(=O)O)c1=O)C[C@@H]1O[C@H]3CC[C@H](C3)N1C2=O ; O=C(NCc1ccc(F)cc1)c1cn2c(c(O)c1=O)C(=O)N1[C@@H]3CC[C@@H](C3)O[C@H]1C2]

### Step 1

1-(2,2-dimethoxyethyl)-5-methoxy-6-(methoxycarbonyl)-4-oxo-1,4-dihydropyridine-3-carboxylic acid (1-A, 3.15 g, 10.0 mmol), suspended in acetonitrile (36 mL) and acetic acid (4 mL) was treated with methane sulfonic acid (0.195 mL). The mixture heated to 75° C. After 7 h, the crude mixture was cooled and stored in a −10° C. for three days. The crude mixture was reheated to 75° C. for 2 h, cooled used without purification in the next step. LCMS-ESI⁺ (m/z): [M+H]⁺ calculated for C₁₉H₂₁F₂N₂O₇: 288.07. found: 288.1.

### Step 2

Crude 1-(2,2-dihydroxyethyl)-5-methoxy-6-(methoxycarbonyl)-4-oxo-1,4-dihydropyridine-3-carboxylic acid (16.8 mL of crude mixture from Step 1, approx 4 mmol) was combined with (1S,3R)-3-aminocyclopentanol (0.809 g, 8 mmol), diluted with acetonitrile (16.8 mL), and treated with potassium carbonate (0.553 g, 4 mmol). The reaction mixture was heated to 85 OC, stirred for 15 min, cooled to ambient temperature and stirred an additional 16 h. HCl (50 mL, 0.2M aq) was added and the clear yellow solution was extracted three times with dichloromethane. The combined organics were dried over sodium sulfate, filtered and concentrated to a yellow solid. This crude material was precipitated from dichloromethane/hexanes to afford desired intermediate 15-B as a light beige powder. ¹H NMR (400 MHz, DMSO-d₆) δ 8.72 (s, 1H), 5.42 (dd, J=9.6, 4.1 Hz, 1H), 5.09 (s, 1H), 4.72 (dd, J=13.0, 3.7 Hz, 1H), 4.57 (s, 1H), 4.09 (dd, J=12.5, 9.6 Hz, 1H), 3.83 (s, 3H), 1.92 (s, 3H), 1.78 (m, 2H), 1.62-1.47 (m, 1H). LCMS-ESI⁺ (m/z): [M+H]⁺ calculated for C₁₅H₁₇N₂O₆: 321.11. found: 321.2.

### Step 3

Intermediate 15-B (0.040 g, 0.125 mmol) and (4-fluorophenyl)methanamine (0.017 g, 0.137 mmol) were suspended in acetonitrile (1 mL) and treated with diisopropylethylamine (0.033 mL, 0.187 mmol) and HATU (0.052 g, 0.137 mmol). After stirring for 30 min, the reaction mixture was treated with magnesium bromide (0.046 g, 0.25 mmol) and heated to 50° C. After 10 min, the reaction mixture was cooled and treated with HCl (2 mL, 10% aq). After a few minutes, the precipitate was filtered and washed with HCl (10% aq) and water. Preparative HPLC purification of the precipitate (20-65% acetonitrile:water, 0.1% TFA) afforded desired Compound 15. ¹H NMR (400 MHz, DMSO-d₆) δ 12.44 (s, 1H), 10.36 (t, J=6.0 Hz, 1H), 8.46 (s, 1H), 7.37-7.28 (m, 2H), 7.19-7.09 (m, 2H), 5.43 (dd, J=9.6, 4.0 Hz, 1H), 5.08 (s, 1H), 4.68 (dd, J=12.8, 4.1 Hz, 1H), 4.59 (s, 1H), 4.58-4.42 (m, 3H), 4.02 (dd, J=12.7, 9.6 Hz, 1H), 1.92 (s, 5H), 1.83 (d, J=12.2 Hz, 1H), 1.56 (dt, J=12.0, 3.4 Hz, 1H). LCMS-ESI⁺ (m/z): [M+H]⁺ calculated for C₂₁H₂₁FN₃O₅:414.15. found: 414.2.

### Example 16

### Preparation of Compound 16

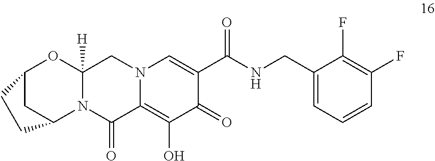 [SMILES: O=C(NCc1cccc(F)c1F)c1cn2c(c(O)c1=O)C(=O)N1[C@@H]3CC[C@@H](C3)O[C@H]1C2]

Compound 16 was prepared in a similar manner to compound 15 using (2,3-difluorophenyl)methanamine in place of (4-fluorophenyl)methanamine. ¹H-NMR ¹H NMR (400 MHz, DMSO-d₆) δ 12.46 (s, 1H), 10.41 (t, J=6.1 Hz, 1H), 8.45 (s, 1H), 7.43-7.25 (m, 1H), 7.25-7.05 (m, 2H), 5.44 (dd, J=9.5, 3.9 Hz, 1H), 5.09 (s, 1H), 4.68 (dd, J=12.8, 4.0 Hz, 1H), 4.65-4.53 (m, 3H), 4.02 (dd, J=12.7, 9.8 Hz, 1H), 3.56 (s, 1H), 1.93 (s, 4H), 1.83 (d, J=11.9 Hz, 1H), 1.57 (dt, J=11.5, 3.0 Hz, 1H). LCMS-ESI⁺ (m/z): [M+H]⁺ calculated for C₂₁H₂₀F₂N₃O₅:432.14. found: 432.2.

### Example 17

### Preparation of Compound 17

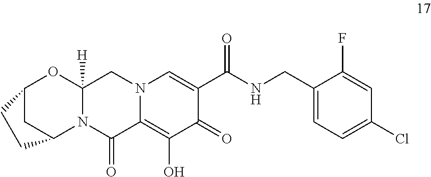 [SMILES: O=C(NCc1ccc(Cl)cc1F)c1cn2c(c(O)c1=O)C(=O)N1[C@@H]3CC[C@@H](C3)O[C@H]1C2]

Compound 17 was prepared in a similar manner to compound 15 using (4-chloro-2-fluorophenyl)methanamine in place of (4-fluorophenyl)methanamine. ¹H-NMR ¹H NMR (400 MHz, DMSO-d₆) δ 12.46 (s, 1H), 10.45-10.29 (m, 1H), 8.44 (s, 1H), 7.42 (dd, J=10.0, 2.0 Hz, 1H), 7.33 (t, J=8.1 Hz, 1H), 7.26 (dd, J=8.4, 1.8 Hz, 1H), 5.50-5.38 (m, 1H), 5.09 (s, 1H), 4.68 (dd, J=13.0, 4.0 Hz, 1H), 4.59 (s, 1H), 4.54 (m, 2H), 4.02 (dd, J=12.8, 9.7 Hz, 1H), 1.93 (s, 4H), 1.83 (d, J=12.0 Hz, 1H), 1.57 (dt, J=11.9, 3.4 Hz, 1H). LCMS-ESI⁺ (m/z): [M+H]⁺ calculated for C₂₁H₂₀ClFN₃O₅:448.11. found: 448.2.

### Example 18

### Preparation of Compound 18

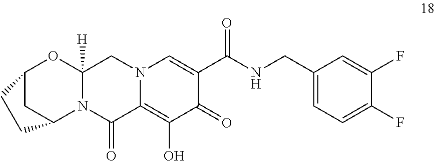 [SMILES: O=C(NCc1ccc(F)c(F)c1)c1cn2c(c(O)c1=O)C(=O)N1[C@@H]3CC[C@@H](C3)O[C@H]1C2]

Compound 18 was prepared in a similar manner to compound 15 using (3,4-difluorophenyl)methanamine in place of (4-fluorophenyl)methanamine. ¹H-NMR (400 MHz, DMSO-d₆) δ 12.46 (s, 1H), 10.51-10.27 (m, 1H), 8.46 (s, 1H), 7.50-7.23 (m, 2H), 7.23-7.03 (m, 1H), 5.44 (dd, J=9.5, 3.6 Hz, 1H), 5.09 (s, 1H), 4.75-4.63 (m, 1H), 4.60 (s, 1H), 4.57-4.44 (m, 2H), 4.02 (dd, J=12.6, 9.8 Hz, 1H), 1.93 (s, 4H), 1.83 (d, J=12.0 Hz, 1H), 1.57 (dt, J=12.0, 3.4 Hz, 1H). LCMS-ESI⁺ (m/z): [M+H]⁺ calculated for C₂₁H₂₀F₂N₃O₅:432.14. found: 432.2.

### Example 19

### Preparation of Compound 19

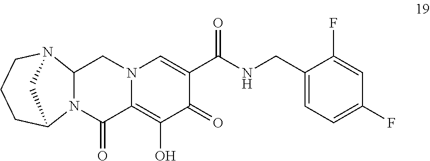 [SMILES: O=C(NCc1ccc(F)cc1F)c1cn2c(c(O)c1=O)C(=O)N1C(C2)[N@]2CCC[C@H]1C2]

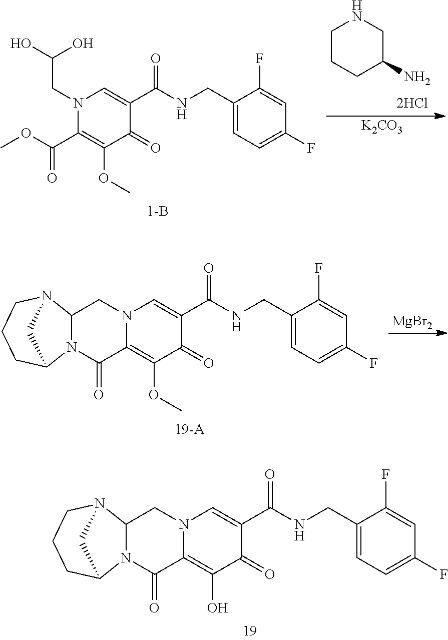

### Steps 1 and 2

Methyl 5-(2,4-difluorobenzylcarbamoyl)-1-(2,2-dihydroxyethyl)-3-methoxy-4-oxo-1,4-dihydropyridine-2-carboxylate (1-B, 97.5 mg, 0.236 mmol) was treated with acetonitrile (1.9 mL), acetic acid (0.1 mL), potassium carbonate (145 mg, 1.05 mmol), and (S)-piperidin-3-amine dihydrochloride (82 mg, 0.472 mmol). The reaction mixture was sealed and heated to 90° C. After 60 minutes, the reaction mixture was cooled partitioned between brine and dichloromethane. The aqueous phase was thrice extracted into dichloromethane and the combined organic phases were combined, dried over MgSO4, filtered, concentrated. The crude product was dissolved into acetonitrile (2 mL) and magnesium bromide (89.1 mg, 0.48 mmol) was added. The mixture was resealed and heated to 50° C. After 90 minutes, the reaction mixture was quenched with ˜5 mL of 0.2M HCl(aq), the pH adjusted to ˜10, diluted with brine, and thrice extracted into DCM. HPLC purification (Acetonitrile:water, 0.1% TFA) afforded Compound 19. ¹H-NMR (400 MHz, Chloroform-d) δ 10.43 (t, J=5.9 Hz, 1H), 8.43 (s, 1H), 7.39-7.30 (m, 1H), 6.81 (q, J=8.1 Hz, 2H), 4.89 (dd, J=11.6, 3.8 Hz, 1H), 4.69 (s, 1H), 4.64 (d, J=5.8 Hz, 2H), 4.26 (dd, J=12.6, 3.8 Hz, 1H), 3.91 (t, J=12.1 Hz, 1H), 3.20-3.10 (m, 2H), 3.06 (s, 2H), 2.14-2.02 (m, 1H), 1.96-1.81 (m, 2H), 1.81-1.70 (m, 1H). LCMS-ESI⁺ (m/z): [M+H]⁺ calculated for C₂₁H₂₀F₂N₄O₄: 431.15. found: 431.2.

### Example 20

### Preparation of Compound 20

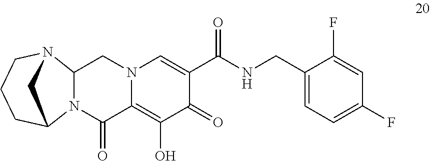 [SMILES: O=C(NCc1ccc(F)cc1F)c1cn2c(c(O)c1=O)C(=O)N1C(C2)[N@@]2CCC[C@@H]1C2]

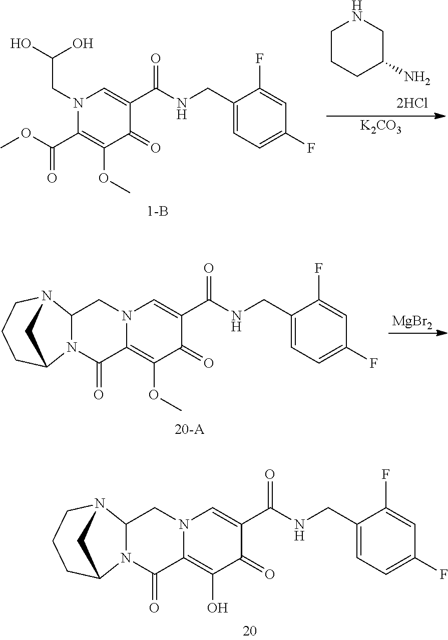

### Steps 1 and 2

Methyl 5-(2,4-difluorobenzylcarbamoyl)-1-(2,2-dihydroxyethyl)-3-methoxy-4-oxo-1,4-dihydropyridine-2-carboxylate (1-B, 103.3 mg, 0.25 mmol) was treated with acetonitrile (1.9 mL), acetic acid (0.1 mL), potassium carbonate (159.8 mg, 1.16 mmol), and (R)-piperidin-3-amine dihydrochloride (90 mg, 0.52 mmol). The reaction mixture was sealed and heated to 90° C. After 40 minutes, the reaction mixture was cooled partitioned between brine and dichloromethane. The aqueous phase was thrice extracted into dichloromethane and the combined organic phases were combined, dried over MgSO4, filtered, concentrated. The crude product was dissolved into acetonitrile (2 mL) and magnesium bromide (96.5 mg, 0.52 mmol) was added. The mixture was resealed and heated to 50° C. After 80 minutes, the reaction mixture was quenched with ˜5 mL of 0.2M HCl(aq), the pH adjusted to ˜10, diluted with brine, and thrice extracted into DCM. HPLC purification (Acetonitrile:water, 0.1% TFA) afforded Compound 20. ¹H-NMR (400 MHz, DMSO-d₆) δ 10.35 (t, J=6.0 Hz, 1H), 8.48 (s, 1H), 7.45-7.33 (m, 1H), 7.29-7.18 (m, 1H), 7.05 (td, J=8.5, 2.4 Hz, 1H), 5.06 (dd, J=11.4, 3.5 Hz, 1H), 4.56-4.47 (m, 3H), 4.44 (s, 1H), 4.05 (t, J=11.8 Hz, 1H), 3.07-2.89 (m, 4H), 1.85-1.73 (m, 3H), 1.54-1.46 (m, 1H). LCMS-ESI⁺ (m/z): [M+H]⁺ calculated for C₂₁H₂₀F₂N₄O₄: 431.15. found: 431.2.

### Example 21

### Preparation of Compound 21

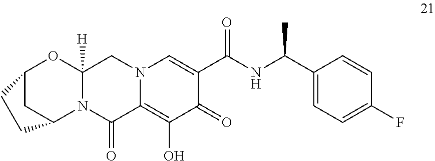 [SMILES: C[C@H](NC(=O)c1cn2c(c(O)c1=O)C(=O)N1[C@@H]3CC[C@@H](C3)O[C@H]1C2)c1ccc(F)cc1]

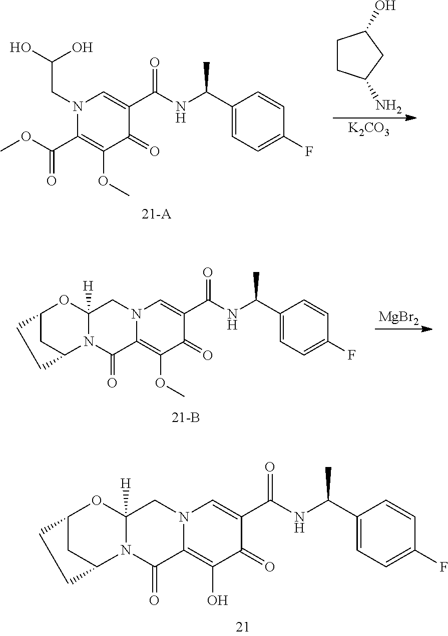

### Steps 1 and 2

(S)-Methyl 1-(2,2-dihydroxyethyl)-5-(1-(4-fluorophenyl)ethylcarbamoyl)-3-methoxy-4-oxo-1,4-dihydropyridine-2-carboxylate (21-A, 1 mL, 0.23 M solution in 19:1 acetonitrile:acetic acid, prepared as per (R)-methyl 1-(2,2-dihydroxyethyl)-5-(1-(4-fluorophenyl)ethylcarbamoyl)-3-methoxy-4-oxo-1,4-dihydropyridine-2-carboxylate from Example AA using (S)-1-(4-fluorophenyl)ethanamine in place of (R)-1-(4-fluorophenyl)ethanamine) was treated with (1S,3R)-3-aminocyclopentanol (62 mg, 0.61 mmol) and potassium carbonate (34 mg, 0.25 mmol). The reaction mixture was sealed and heated to 90° C. After 60 minutes, the reaction mixture was cooled partitioned between brine and dichloromethane. The aqueous phase was thrice extracted into dichloromethane and the combined organic phases were combined, dried over MgSO4, filtered, and concentrated. The crude product was dissolved into acetonitrile (2 mL) and magnesium bromide (74 mg, 0.4 mmol) was added. The mixture was resealed and heated to 50° C. After 100 minutes, the reaction mixture was quenched with 0.2M HCl(aq), diluted with brine, and thrice extracted into DCM. HPLC purification (Acetonitrile:water, 0.1% TFA) afforded Compound 21. ¹H-NMR (400 MHz, DMSO-d₆) δ 12.42 (br s, 1H), 10.45 (d, J=7.9 Hz, 1H), 8.40 (s, 1H), 7.36 (dd, J=8.6, 5.5 Hz, 2H), 7.14 (t, J=8.9 Hz, 2H), 5.42 (dd, J=9.6, 4.2 Hz, 1H), 5.15-5.04 (m, 2H), 4.72-4.55 (m, 2H), 4.02 (dd, J=12.7, 9.7 Hz, 1H), 1.97-1.89 (m, 4H), 1.82 (d, J=12.2 Hz, 1H), 1.56 (dt, J=11.9, 3.3 Hz, 1H), 1.43 (d, J=6.9 Hz, 3H). LCMS-ESI⁺ (m/z): [M+H]⁺ calculated for C₂₂H₂₂FN₃O₅:428.16. found: 428.1.

### Example 22

### Preparation of Compound 22

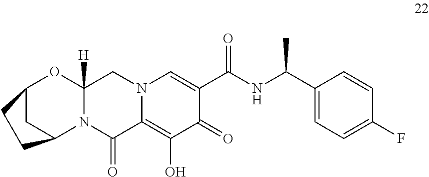 [SMILES: C[C@H](NC(=O)c1cn2c(c(O)c1=O)C(=O)N1[C@H]3CC[C@H](C3)O[C@@H]1C2)c1ccc(F)cc1]

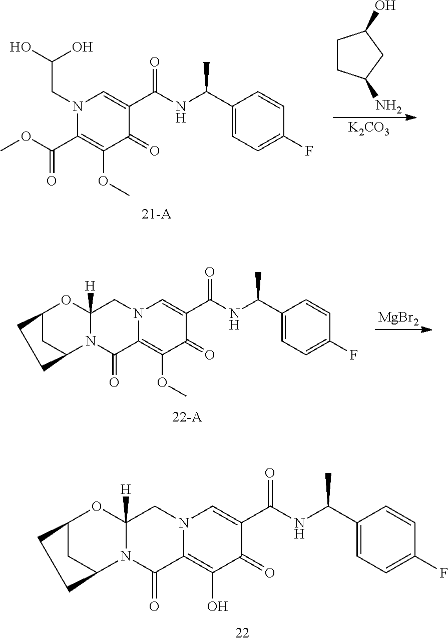

### Steps 1 and 2

(S)-methyl 1-(2,2-dihydroxyethyl)-5-(1-(4-fluorophenyl)ethylcarbamoyl)-3-methoxy-4-oxo-1,4-dihydropyridine-2-carboxylate (21-A, 1 mL, 0.23 M solution in 19:1 acetonitrile:acetic acid) was treated with (1R,3S)-3-aminocyclopentanol (52 mg, 0.51 mmol) and potassium carbonate (31 mg, 0.22 mmol). The reaction mixture was sealed and heated to 90° C. After 60 minutes, the reaction mixture was cooled partitioned between brine and dichloromethane. The aqueous phase was thrice extracted into dichloromethane and the combined organic phases were combined, dried over MgSO4, filtered, and concentrated. The crude product was dissolved into acetonitrile (2 mL) and magnesium bromide (91 mg, 0.49 mmol) was added. The mixture was resealed and heated to 50° C. After 100 minutes, the reaction mixture was quenched with 0.2M HCl(aq), diluted with brine, and thrice extracted into DCM. HPLC purification (Acetonitrile:water, 0.1% TFA) afforded Compound 22. ¹H-NMR (400 MHz, DMSO-d₆) δ 12.44 (br s, 1H), 10.45 (d, J=7.7 Hz, 1H), 8.39 (s, 1H), 7.36 (dd, J=8.5, 5.6 Hz, 2H), 7.14 (t, J=8.9 Hz, 2H), 5.43 (dd, J=9.6, 4.0 Hz, 1H), 5.15-5.06 (m, 2H), 4.66 (dd, J=12.8, 3.9 Hz, 1H), 4.58 (s, 1H), 3.99 (dd, J=12.6, 9.5 Hz, 1H), 1.93 (s, 4H), 1.82 (d, J=12.0 Hz, 1H), 1.56 (dt, J=12.0, 3.0 Hz, 1H), 1.44 (d, J=6.9 Hz, 3H). LCMS-ESI⁺ (m/z): [M+H]⁺ calculated for C₂₂H₂₂FN₃O₅: 428.16. found: 428.1.

### Example 23

### Preparation of Compound 23

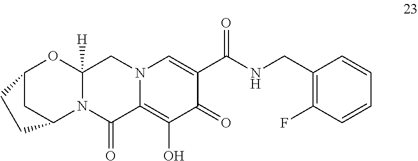 [SMILES: O=C(NCc1ccccc1F)c1cn2c(c(O)c1=O)C(=O)N1[C@@H]3CC[C@@H](C3)O[C@H]1C2]

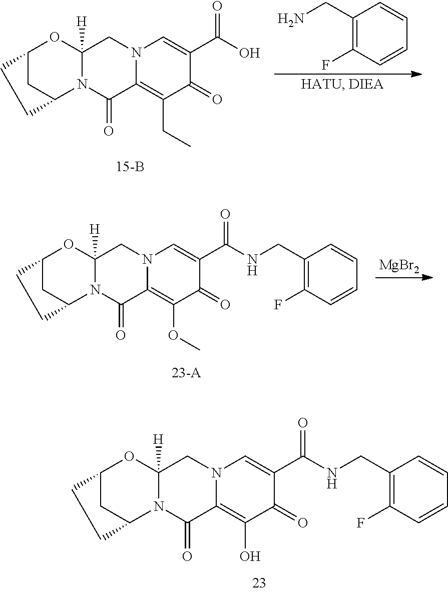

### Steps 1 and 2

15-B (41 mg, 0.13 mmol) was treated with acetonitrile (1 mL), (2-fluorophenyl)methanamine (17 mg, 0.14 mmol), HATU (67 mg, 0.18 mmol), and N,N-diisopropylethylamine (24 mg, 0.19 mmol). The reaction mixture was stirred at room temperature for one hour and magnesium bromide (47 mg, 0.26 mmol) was added. The mixture was sealed and heated to 50° C. After 60 minutes, the reaction mixture was quenched with 0.2M HCl(aq), diluted with brine, and thrice extracted into DCM. HPLC purification (Acetonitrile:water, 0.1% TFA) afforded Compound 23. ¹H-NMR (400 MHz, Chloroform-d) δ 10.42 (s, 1H), 8.34 (s, 1H), 7.36 (t, J=7.9 Hz, 1H), 7.24-7.17 (m, 1H), 7.12-6.97 (m, 2H), 5.40-5.32 (m, 1H), 5.29 (t, J=3.5 Hz, 1H), 4.67 (s, 3H), 4.28-4.20 (m, 1H), 4.06-3.95 (m, 1H), 2.20-1.96 (m, 4H), 1.95-1.84 (m, 1H), 1.59 (dt, J=12.4, 3.3 Hz, 1H). LCMS-ESI⁺ (m/z): [M+H]⁺ calculated for C₂₁H₂₀FN₃O₅:414.15. found: 414.2.

### Example 24

### Preparation of Compound 24

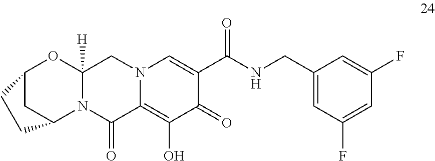 [SMILES: O=C(NCc1cc(F)cc(F)c1)c1cn2c(c(O)c1=O)C(=O)N1[C@@H]3CC[C@@H](C3)O[C@H]1C2]

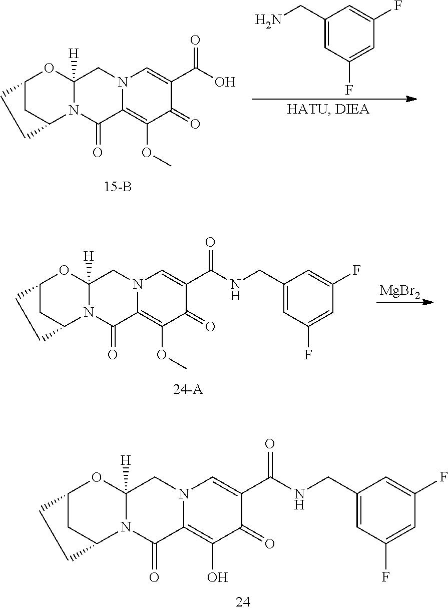

### Steps 1 and 2

15-B (44 mg, 0.14 mmol) was treated with acetonitrile (1 mL), (3,5-difluorophenyl)methanamine (32 mg, 0.23 mmol), HATU (54 mg, 0.14 mmol), and N,N-diisopropylethylamine (37 mg, 0.29 mmol). The reaction mixture was stirred at room temperature for one hour and magnesium bromide (57 mg, 0.31 mmol) was added. The mixture was sealed and heated to 50° C. After 60 minutes, the reaction mixture was quenched with 0.2M HCl(aq), diluted with brine, and thrice extracted into DCM. HPLC purification (Acetonitrile:water, 0.1% TFA) afforded Compound 24. ¹H-NMR (400 MHz, Chloroform-d) δ 10.39 (s, 1H), 8.42 (s, 1H), 6.82 (d, J=7.9 Hz, 2H), 6.65 (t, J=8.8 Hz, 1H), 5.38 (d, J=7.7 Hz, 1H), 5.28 (s, 1H), 4.78-4.41 (m, 3H), 4.32 (d, J=12.1 Hz, 1H), 4.02 (t, J=10.9 Hz, 1H), 2.30-1.97 (m, 4H), 1.97-1.81 (m, 1H), 1.59 (d, J=12.3 Hz, 1H). LCMS-ESI⁺ (m/z): [M+H]⁺ calculated for C₂₁H₁₉F₂N₃O₅:432.14. found: 432.2.

### Example 25

### Preparation of Compound 25

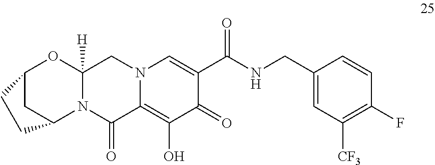 [SMILES: O=C(NCc1ccc(F)c(C(F)(F)F)c1)c1cn2c(c(O)c1=O)C(=O)N1[C@@H]3CC[C@@H](C3)O[C@H]1C2]

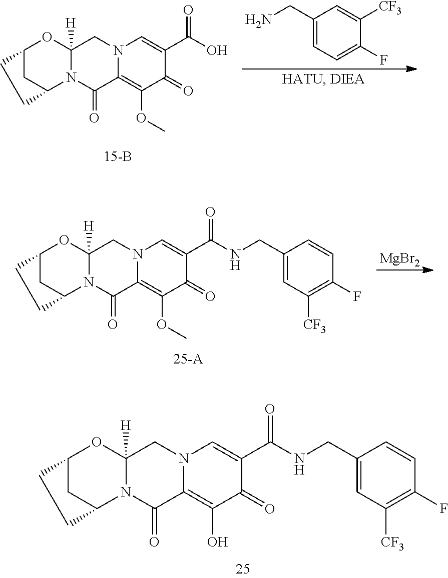

### Steps 1 and 2

15-B (43 mg, 0.13 mmol) was treated with acetonitrile (1 mL), (4-fluoro-3-(trifluoromethyl)phenyl)methanamine (29 mg, 0.15 mmol), HATU (62 mg, 0.16 mmol), and N,N-diisopropylethylamine (26 mg, 0.20 mmol). The reaction mixture was stirred at room temperature for one hour and magnesium bromide (62 mg, 0.34 mmol) was added. The mixture was sealed and heated to 50° C. After 60 minutes, the reaction mixture was quenched with 0.2M HCl(aq), diluted with brine, and thrice extracted into DCM. HPLC purification (Acetonitrile:water, 0.1% TFA) afforded Compound 25. ¹H-NMR (400 MHz, Chloroform-d) δ 10.44 (s, 1H), 8.29 (s, 1H), 7.56-7.38 (m, 2H), 7.06 (t, J=9.2 Hz, 1H), 5.30 (dd, J=9.3, 3.5 Hz, 1H), 5.21 (s, 1H), 4.65-4.45 (m, 3H), 4.21 (dd, J=12.8, 3.4 Hz, 1H), 3.95 (dd, J=12.4, 9.7 Hz, 1H), 2.11-1.89 (m, 4H), 1.89-1.74 (m, 1H), 1.53 (dt, J=12.4, 3.2 Hz, 1H). LCMS-ESI⁺ (m/z): [M+H]⁺ calculated for C₂₂H₁₉F₄N₃O₅:482.14. found: 482.2.

### Example 26

### Preparation of Compound 26

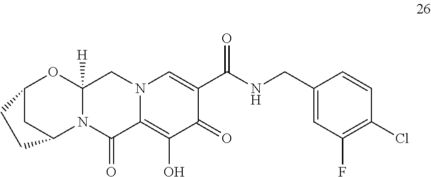 [SMILES: O=C(NCc1ccc(Cl)c(F)c1)c1cn2c(c(O)c1=O)C(=O)N1[C@@H]3CC[C@@H](C3)O[C@H]1C2]

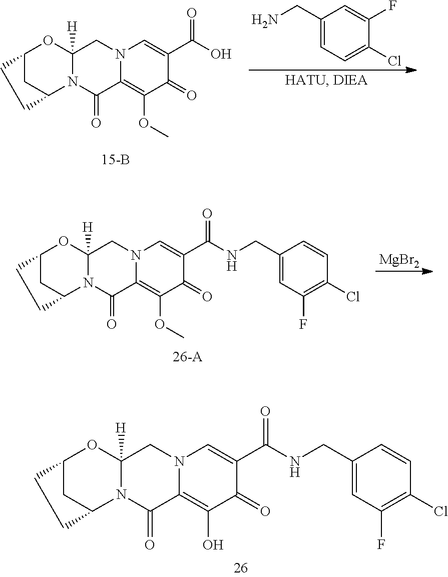

### Steps 1 and 2

15-B (41 mg, 0.13 mmol) was treated with acetonitrile (1 mL), (4-chloro-3-fluorophenyl)methanamine (40 mg, 0.25 mmol), HATU (60 mg, 0.16 mmol), and N,N-diisopropylethylamine (28 mg, 0.22 mmol). The reaction mixture was stirred at room temperature for one hour and magnesium bromide (48 mg, 0.26 mmol) was added. The mixture was sealed and heated to 50° C. After 60 minutes, the reaction mixture was quenched with 0.2M HCl(aq), diluted with brine, and thrice extracted into DCM. HPLC purification (Acetonitrile:water, 0.1% TFA) afforded Compound 26. ¹H-NMR (400 MHz, Chloroform-d) δ 10.41 (s, 1H), 8.30 (s, 1H), 7.24 (t, J=6.1 Hz, 1H), 7.13-6.90 (m, 2H), 5.30 (dd, J=9.1, 3.2 Hz, 1H), 5.22 (s, 1H), 4.61 (s, 1H), 4.51 (s, 2H), 4.20 (d, J=9.4 Hz, 1H), 3.95 (d, J=12.0 Hz, 1H), 2.11-1.90 (m, 4H), 1.90-1.76 (m, 1H), 1.53 (d, J=12.2 Hz, 1H). LCMS-ESI⁺ (m/z): [M+H]⁺ calculated for C₂₁H₁₉ClFN₃O₅:448.11. found: 448.2.

### Example 27

### Preparation of Compound 27

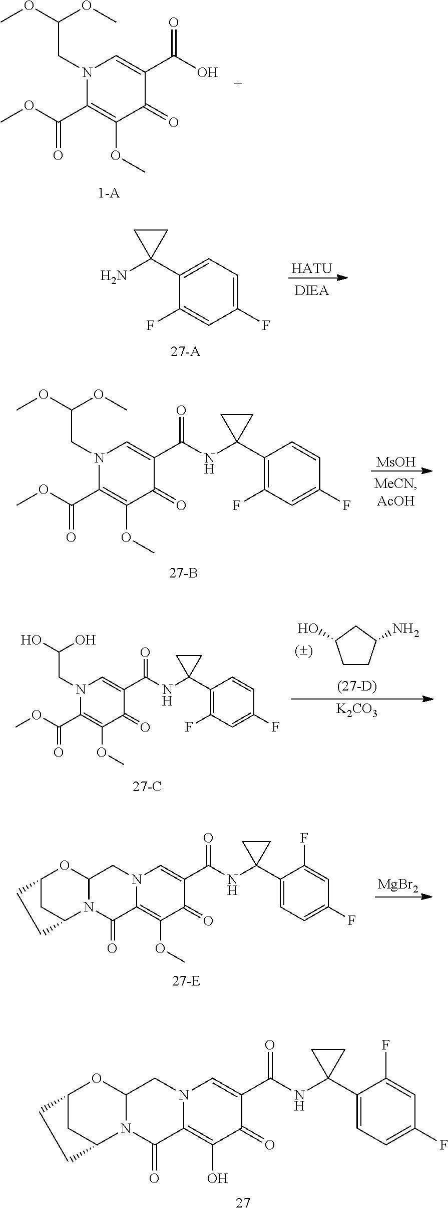

### Step 1

A suspension of the compound 1-A (1.004 g, 3.19 mmol), the amine 27-A (688 mg, 3.35 mmol), and HATU (1.453 g 3.82 mmol) in CH₂Cl₂ (20 mL) was stirred in 0° C. bath as DIEA (2 mL, 11.48 mmol) was added. After 1 h at 0° C., the reaction mixture was concentrated to a syrup, diluted with ethyl acetate, and washed with water (×2). After the aq. fractions were extracted with ethyl acetate (×1), the organic fractions were combined, dried (Na₂SO₄), and concentrated. The residue was purified by CombiFlash (120 g column) using hexanes-ethyl acetate as eluents. The major peak was combined and concentrated to get 1.082 g (73%) of the product 27-B. After the minor peak was combined and concentrated, the concentrated residue was dissolved in CH₂Cl₂ and some insoluble materials were filtered. The filtrate was concentrated to get 361 mg (24%) of the additional product 27-B. LCMS-ESI⁺ (m/z): [M+H]⁺ calculated for C₂₂H₂₅F₂N₂O₇: 467.16. found: 467.1.

### Step 2 and 3

Compound 27-B (81 mg, 0.174 mmol) was dissolved in a mixture (1 mL) of acetonitrile (22 mL), AcOH (2 mL), and methanesulfonic acid (0.14 mL, 2.16 mmol) at rt and the resulting solution was stirred at 65° C. for 20 h.

After the resulting solution was cooled to rt, the aminoalcohol 27-D (50 mg, racemic, 0.363 mmol), K₂CO₃ (50 mg, 0.362 mmol), and acetonitrile (2 mL) were added to the solution. The resulting mixture was stirred at 65° C. bath for 1 h. After the reaction mixture was cooled to rt, it was acidified with 1 N HCl (˜2 mL), diluted with water (˜8 mL), and extracted with CH₂Cl₂ (×3). Combined extracts were dried (Na₂SO₄), concentrated, and purified by CombiFlash to obtain 67 mg (82%) of compound 27-E. ¹H NMR (400 MHz, CDCl₃) δ 10.53 (s, 1H), 8.25 (s, 1H), 7.60 (td, J=8.5, 6.5 Hz, 1H), 6.85-6.57 (m, 2H), 5.33 (br, 1H), 5.26 (dd, J=9.6, 3.9 Hz, 1H), 4.60 (t, J=3.0 Hz, 1H), 4.18-4.06 (m, 1H), 4.01 (s, 3H), 3.92 (dd, J=12.7, 9.6 Hz, 1H), 2.11-1.91 (m, 4H), 1.88-1.71 (m, 1H), 1.60-1.49 (m, 1H), 1.31-1.10 (m, 4H). ¹⁹F NMR (376.1 MHz, CDCl₃) δ −111.80 (q, J=8.8 Hz, 1F), −112.05 (p, J=7.9 Hz, 1F). LCMS-ESI⁺ (m/z): [M+H]⁺ calculated for C₂₄H₂₄F₂N₃O₅:472.17. found: 472.1.

### Step 4

A mixture of compound 27-E (67 mg, 0.142 mmol) and MgBr₂ (66 mg, 0.358 mmol) in MeCN (3 mL) was stirred at 50° C. for 30 min and cooled to 0° C. before treating with 1 N HCl (3 mL). After the mixture was diluted with water (˜30 mL), the product was extracted with CH₂Cl₂ (×3), and the combined extracts were dried (Na₂SO₄) and concentrated. The product was purified by preparative HPLC and freeze-dried to obtain 58 mg (71%) of the product 27 as a 1:1 mixture with trifluoroacetic acid. ¹H NMR (400 MHz, CDCl₃) δ 10.70 (s, 1H), 8.35 (s, 1H), 7.57 (q, J=8.2 Hz, 1H), 6.91-6.56 (m, 2H), 5.31 (dt, J=14.3, 4.0 Hz, 2H), 4.68 (s, 1H), 4.22 (dd, J=13.2, 3.9 Hz, 1H), 3.99 (dd, J=12.8, 9.3 Hz, 1H), 2.28-1.96 (m, 5H), 1.88 (ddt, J=12.1, 8.6, 3.7 Hz, 1H), 1.71-1.49 (m, 1H), 1.38-1.11 (m, 4H). ¹⁹F NMR (376.1 MHz, CDCl₃) δ −76.37 (s, 3F), −111.6˜−111.75 (m, 2F). LCMS-ESI⁺ (m/z): [M+H]⁺ calculated for C₂₃H₂₂F₂N₃O₅:458.15. found: 458.1.

### Example 28

### Preparation of Compound 28

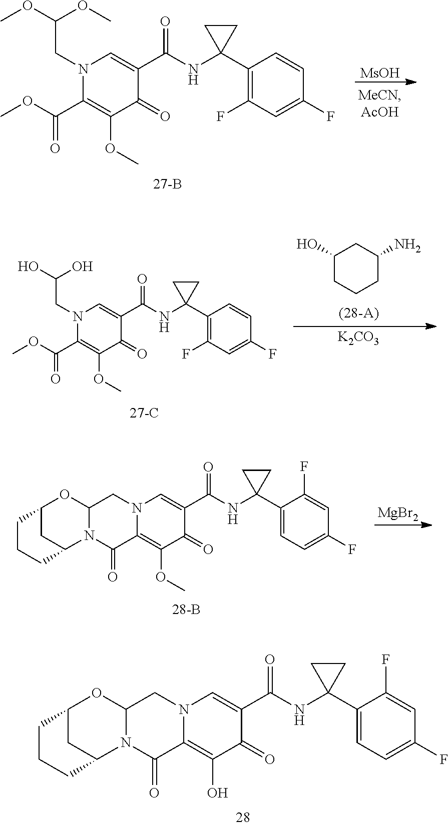

### Step 1 and 2

Compound 27-B (87 mg, 0.187 mmol) was dissolved in a mixture (2 mL) of acetonitrile (22 mL), AcOH (2 mL), and methanesulfonic acid (0.14 mL, 2.16 mmol) at rt and the resulting solution was stirred at 65° C. for 20 h.

After the resulting solution was cooled to rt, the aminoalcohol 28-A (44 mg, racemic, 0.382 mmol) and acetonitrile (2 mL) were added to the solution. After the resulting mixture was stirred at 65° C. bath for 30 min, K₂CO₃ (41 mg, 0.297 mmol) was added and the mixture was stirred at 65° C. for 21 h. The reaction mixture was cooled to rt, it was acidified with 1 N HCl (˜2 mL), diluted with water (˜8 mL), and extracted with CH₂Cl₂ (×3). Combined extracts were dried (Na₂SO₄), concentrated, and purified by preparative HPLC and the fraction containing the product was freeze-dried. After the residue was dissolved in EA, the solution was washed with saturated NaHCO₃ (×1), dried (Na₂SO₄), and concentrated to obtain 18 mg (20%) of compound 28-B as a 1:1 mixture with trifluoroacetic acid. ¹H NMR (400 MHz, CDCl₃) δ 10.54 (s, 1H), 8.26 (s, 1H), 7.63 (td, J=8.6, 6.6 Hz, 1H), 6.76 (dddd, J=21.9, 11.2, 8.7, 2.3 Hz, 2H), 5.39 (dd, J=9.6, 3.7 Hz, 1H), 4.53-4.36 (m, 2H), 4.09 (dd, J=12.8, 3.7 Hz, 1H), 4.03 (s, 3H), 3.99 (dd, J=12.7, 9.7 Hz, 1H), 2.41-2.20 (m, 2H), 1.84 (dtd, J=19.7, 9.3, 8.8, 4.4 Hz, 2H), 1.74 (dd, J=14.6, 2.5 Hz, 1H), 1.62-1.35 (m, 2H), 1.34-1.14 (m, 5H). ¹⁹F NMR (376.1 MHz, CDCl₃) δ −111.75 (q, J=8.9 Hz, 1F), −112.01 (p, J=7.9 Hz, 1F). LCMS-ESI⁺ (m/z): [M+H]⁺ calculated for C₂₅H₂₆F₂N₃O₅:486.18. found: 486.2.

### Step 3

Compound 28-B (18 mg, 0.037 mmol) was treated with MgBr₂ as described in step 4 in the synthesis of compound 27-E to obtain 12 mg (55%) of compound 28. ¹H NMR (400 MHz, CDCl₃) δ 10.66 (s, 1H), 8.29 (s, 1H), 7.59 (td, J=8.5, 6.6 Hz, 1H), 6.89-6.60 (m, 2H), 5.51 (dd, J=9.9, 4.0 Hz, 1H), 4.55 (s, 1H), 4.48 (t, J=4.2 Hz, 1H), 4.21 (dd, J=12.9, 4.1 Hz, 1H), 3.99 (dd, J=12.8, 9.8 Hz, 1H), 2.56-2.35 (m, 1H), 2.14 (dd, J=16.1, 5.9 Hz, 1H), 1.96-1.74 (m, 3H), 1.66-1.37 (m, 3H), 1.28 (d, J=4.4 Hz, 2H), 1.26-1.19 (m, 2H). ¹⁹F NMR (376.1 MHz, CDCl₃) δ −76.41 (s, 3F, −111.79 (m, 2F). LCMS-ESI⁺ (m/z): [M+H]⁺ calculated for C₂₄H₂₃F₂N₃O₅: 472.17. found: 472.1.

### Example 29

### Preparation of Compound 29

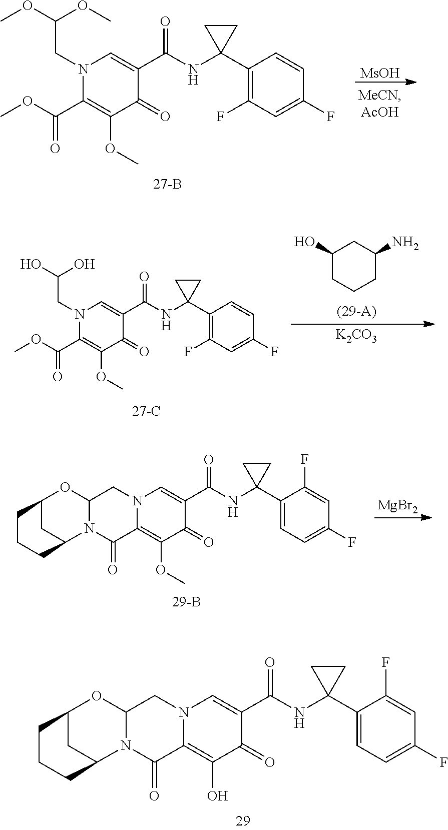

### Step 1 and 2

Compound 29-B (13 mg, 14%) was prepared from compound 27-B (87 mg, 0.187 mmol) and the aminoalcohol 29-A (45 mg, 0.391 mmol) in a manner similar to that described in step 1 of the synthesis of compound 28-B. ¹H NMR (400 MHz, CDCl₃) δ 10.54 (s, 1H), 8.26 (s, 1H), 7.63 (td, J=8.6, 6.6 Hz, 1H), 6.76 (dddd, J=21.9, 11.2, 8.7, 2.3 Hz, 2H), 5.39 (dd, J=9.6, 3.7 Hz, 1H), 4.53-4.36 (m, 2H), 4.09 (dd, J=12.8, 3.7 Hz, 1H), 4.03 (s, 3H), 3.99 (dd, J=12.7, 9.7 Hz, 1H), 2.41-2.20 (m, 2H), 1.84 (dtd, J=19.7, 9.3, 8.8, 4.4 Hz, 2H), 1.74 (dd, J=14.6, 2.5 Hz, 1H), 1.62-1.35 (m, 2H), 1.34-1.14 (m, 5H). ¹⁹F NMR (376.1 MHz, CDCl₃) δ −111.75 (q, J=8.9 Hz, 1F), −112.01 (p, J=7.9 Hz, 1F). LCMS-ESI⁺ (m/z): [M+H]⁺ calculated for C₂₅H₂₆F₂N₃O₅:486.18. found: 486.2.

### Step 3

Compound 29 (8.2 mg, 52%) was prepared from compound 29-B (13 mg, 0.027 mmol) in a manner similar to that described in step 2 of the synthesis of compound 16. ¹H NMR (400 MHz, CDCl₃) δ 10.66 (s, 1H), 8.29 (s, 1H), 7.59 (td, J=8.5, 6.6 Hz, 1H), 6.89-6.60 (m, 2H), 5.51 (dd, J=9.9, 4.0 Hz, 1H), 4.55 (s, 1H), 4.48 (t, J=4.2 Hz, 1H), 4.21 (dd, J=12.9, 4.1 Hz, 1H), 3.99 (dd, J=12.8, 9.8 Hz, 1H), 2.56-2.35 (m, 1H), 2.14 (dd, J=16.1, 5.9 Hz, 1H), 1.96-1.74 (m, 3H), 1.66-1.37 (m, 3H), 1.28 (d, J=4.4 Hz, 2H), 1.26-1.19 (m, 2H). ¹⁹F NMR (376.1 MHz, CDCl₃) δ −76.41 (s, 3F, −111.79 (m, 2F). LCMS-ESI⁺ (m/z): [M+H]⁺ calculated for C₂₄H₂₃F₂N₃O₅: 472.17. found: 472.1.

### Example 30

### Preparation of Compound 30

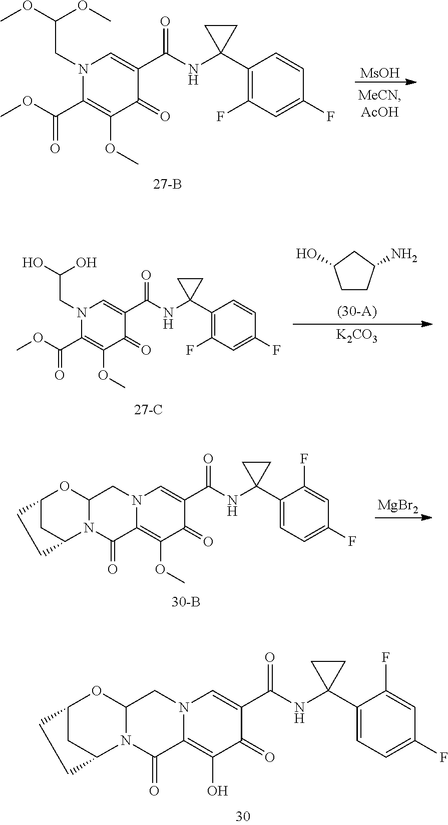

### Step 1 and 2

Compound 27-B (150 mg, 0.322 mmol) was dissolved in acetonitrile (2 mL), AcOH (0.2 mL), and methanesulfonic acid (0.007 mL, 0.108 mmol) at rt and the resulting solution was stirred at 65° C. for 20 h.

After the resulting solution was cooled to rt, the aminoalcohol 30-A (72.1 mg, chiral, 0.713 mmol), K₂CO₃ (89.4 mg, 0.647 mmol), and acetonitrile (2 mL) were added to the solution. The resulting mixture was stirred at 65° C. bath for 0.5 h. After the reaction mixture was cooled to rt, it was acidified with 1 N HCl (˜3 mL), diluted with water (˜12 mL), and extracted with CH₂Cl₂ (×3). Combined extracts were dried (Na₂SO₄), concentrated, and purified by CombiFlash to obtain 128 mg (84%) of compound 30-B. ¹H NMR (400 MHz, CDCl₃) δ 10.52 (s, 1H), 8.24 (s, 1H), 7.61 (td, J=8.6, 6.6 Hz, 1H), 6.85-6.65 (m, 2H), 5.33 (t, J=4.1 Hz, 1H), 5.25 (dd, J=9.5, 3.9 Hz, 1H), 4.61 (d, J=3.4 Hz, 1H), 4.18-4.08 (m, 1H), 4.02 (s, 3H), 3.99-3.87 (m, 1H), 2.12-1.91 (m, 4H), 1.85-1.69 (m, 1H), 1.55 (ddd, J=12.3, 4.1, 2.8 Hz, 1H), 1.31-1.14 (m, 4H). ¹⁹F NMR (376.1 MHz, CDCl₃) δ −111.79 (q, J=8.8 Hz, 1F), −112.05 (p, J=7.9 Hz, 1F). LCMS-ESI⁺ (m/z): [M+H]⁺ calculated for C₂₄H₂₄F₂N₃O₅:472.17. found: 472.2.

### Step 3

A mixture of compound 30-B (128 mg, 0.272 mmol) and MgBr₂ (130 mg, 0.706 mmol) in MeCN (5 mL) was stirred at 50° C. for 30 min and cooled to 0° C. before treating with 1 N HCl (4 mL). After the mixture was diluted with water, the product was extracted with CH₂Cl₂ (×3), and the combined extracts were dried (Na₂SO₄) and concentrated. The product was purified by CombiFlash to obtain 109 mg (88%) of the product 30. ¹H NMR (400 MHz, CDCl₃) δ 12.27 (s, 1H), 10.52 (s, 1H), 8.16 (s, 1H), 7.61 (td, J=8.6, 6.6 Hz, 1H), 6.96-6.54 (m, 2H), 5.36-5.23 (m, 2H), 4.66 (t, J=3.1 Hz, 1H), 4.18-4.06 (m, 1H), 3.94 (dd, J=12.8, 9.4 Hz, 1H), 2.20-1.95 (m, 4H), 1.89 (td, J=11.4, 9.8, 6.7 Hz, 1H), 1.70-1.54 (m, 1H), 1.32-1.15 (m, 4H). ¹⁹F NMR (376.1 MHz, CDCl₃) δ −111.87 (q, J=8.9 Hz, 1F), −112.21 (p, J=7.9 Hz, 1F). LCMS-ESI⁺ (m/z): [M+H]⁺ calculated for C₂₃H₂₂F₂N₃O₅:458.15. found: 458.2.

### Example 31

### Preparation of Compound 31

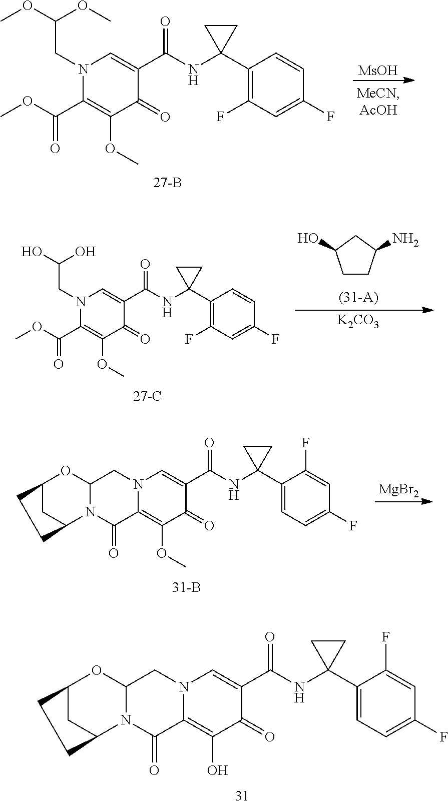

### Step 1 and 2

Compound 31-B (123 mg, 81%) was prepared from compound 27-B (150 mg, 0.322 mmol) and the aminoalcohol 31-A (70.3 mg, 0.695 mmol) in a manner similar to that described in step 1 and 2 of the synthesis of compound 30-B. ¹H NMR (400 MHz, CDCl₃) δ 10.52 (s, 1H), 8.24 (s, 1H), 7.62 (td, J=8.6, 6.6 Hz, 1H), 6.91-6.63 (m, 2H), 5.33 (t, J=4.1 Hz, 1H), 5.25 (dd, J=9.5, 3.9 Hz, 1H), 4.61 (d, J=3.4 Hz, 1H), 4.14-4.07 (m, 1H), 4.03 (s, 3H), 3.93 (dd, J=12.7, 9.5 Hz, 1H), 2.12-1.91 (m, 4H), 1.85-1.69 (m, 1H), 1.55 (ddd, J=12.3, 4.1, 2.8 Hz, 1H), 1.31-1.14 (m, 4H). ¹⁹F NMR (376.1 MHz, CDCl₃) δ −111.79 (q, J=9.2, 8.7 Hz, 1F), −112.03 (h, J=8.1, 7.5 Hz, 1F). LCMS-ESI⁺ (m/z): [M+H]⁺ calculated for C₂₄H₂₄F₂N₃O₅:472.17. found: 472.1.

### Step 3

Compound 31 (88.6 mg, 74%) was prepared from compound 31-B (123 mg, 0.261 mmol) in a manner similar to that described in step 3 of the synthesis of compound 30. ¹H NMR (400 MHz, CDCl₃) δ 12.26 (s, 1H), 10.49 (s, 1H), 8.13 (s, 1H), 7.58 (td, J=8.6, 6.5 Hz, 1H), 6.90-6.56 (m, 2H), 5.32 (dd, J=9.4, 4.1 Hz, 1H), 5.27-5.22 (m, 1H), 4.64 (t, J=3.1 Hz, 1H), 4.11 (dd, J=12.8, 4.0 Hz, 1H), 4.01-3.79 (m, 1H), 2.28-1.95 (m, 4H), 1.95-1.80 (m, 1H), 1.71 (m, 1H), 1.56 (m, 1H), 1.42-1.08 (m, 4H). ¹⁹F NMR (376.1 MHz, CDCl₃) δ −111.95 (q, J=8.9 Hz, 1F), −112.22 (p, J=7.9 Hz, 1F). LCMS-ESI⁺ (m/z): [M+H]⁺ calculated for C₂₃H₂₂F₂N₃O₅:458.15. found: 458.1.

### Example 32

### Preparation of Compound 32

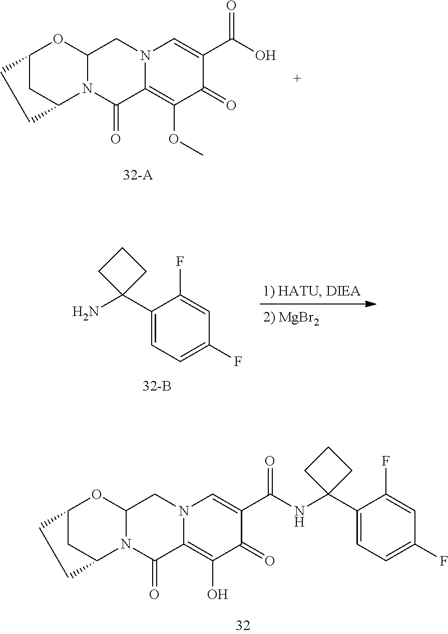 [SMILES: COc1c2n(cc(C(=O)O)c1=O)CC1O[C@H]3CC[C@H](C3)N1C2=O ; NC1(c2ccc(F)cc2F)CCC1 ; O=C(NC1(c2ccc(F)cc2F)CCC1)c1cn2c(c(O)c1=O)C(=O)N1C(C2)O[C@H]2CC[C@@H]1C2]

A solution of compound 32-A (22.2 mg, 0.069 mmol), compound 32-B (18.7 mg, 0.102 mmol), and HATU (43 mg, 0.113 mmol) in CH₂Cl₂ (2 mL) was stirred at rt as DIEA (0.075 mL, 0.431 mmol) was added. After 30 min, the reaction mixture was diluted with ethyl acetate and washed with water (×2). After the aqueous fractions were extracted with EA (×1), the organic fractions were combined, dried, concentrated, and dried in vacuum.

A mixture of the above crude product and MgBr₂ (35 mg, 0.190 mmol) in MeCN (2 mL) was stirred at 50° C. bath for 1 h and cooled to 0° C. before treated with 1 N HCl (˜ 1 mL). The resulting solution was diluted with water, and extracted with CH₂Cl₂ (×3). The combined extracts were dried (Na₂SO₄), and concentrated. The product was purified by preparative HPLC and freeze-dried to obtain 32.7 mg (81%) of compound 32. ¹H NMR (400 MHz, CDCl₃) δ 10.87 (s, 1H), ˜9.3 (br, 1H), 8.35 (s, 1H), 7.50 (td, J=8.7, 6.3 Hz, 1H), 6.89-6.78 (m, 1H), 6.72 (ddd, J=11.2, 8.9, 2.6 Hz, 1H), 5.48-5.12 (m, 2H), 4.72-4.60 (m, 1H), 4.22 (dd, J=13.0, 4.1 Hz, 1H), 3.98 (dd, J=12.9, 9.4 Hz, 1H), 2.68 (m, 4H), 2.33-1.98 (m, 6H), 1.90 (m, 2H), 1.60 (ddd, J=12.4, 4.1, 2.7 Hz, 1H). ¹⁹F NMR (376.1 MHz, CD₃CN) δ −76.39 (s, 3F), −110.50 (q, J=9.2 Hz, 1F), −112.65 (p, J=7.8 Hz, 1F). LCMS-ESI⁺ (m/z): [M+H]⁺ calculated for C₂₄H₂₄F₂N₃O₅:472.17. found: 472.0.

### Example 33

### Preparation of Compound 33

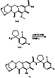 [SMILES: COc1c2n(cc(C(=O)O)c1=O)CC1O[C@H]3CC[C@H](C3)N1C2=O ; NC1(c2ccc(F)cc2F)CCCC1 ; O=C(NC1(c2ccc(F)cc2F)CCCC1)c1cn2c(c(O)c1=O)C(=O)N1C(C2)O[C@H]2CC[C@@H]1C2]

Compound 33 (33.1 mg, 82%) was obtained from compound 32-A (21.8 mg, 0.068 mmol) and compound 33-A (18.7 mg, 0.095 mmol) as described in the synthesis of compound 32. ¹H NMR (400 MHz, CDCl₃) δ 10.70 (s, 1H), ˜9.5 (br, 1H), 8.41 (s, 1H), 7.43 (td, J=8.9, 6.4 Hz, 1H), 6.85-6.76 (m, 1H), 6.72 (ddd, J=11.5, 8.8, 2.6 Hz, 1H), 5.48-5.18 (m, 2H), 4.68 (t, J=3.2 Hz, 1H), 4.26 (dd, J=13.0, 4.1 Hz, 1H), 4.00 (dd, J=13.0, 9.4 Hz, 1H), 2.72-2.45 (m, 2H), 2.22-1.96 (m, 6H), 1.96-1.75 (m, 5H), 1.60 (ddd, J=12.5, 4.1, 2.7 Hz, 1H). ¹⁹F NMR (376.1 MHz, CD₃CN) δ −76.41 (s, 3F), −107.86 (q, J=9.4 Hz, 1F), −113.13 (p, J=8.0 Hz, 1F). LCMS-ESI⁺ (m/z): [M+H]⁺ calculated for C₂₅H₂₆F₂N₃O₅:486.18. found: 485.9.

### Example 34

### Preparation of Compound 34

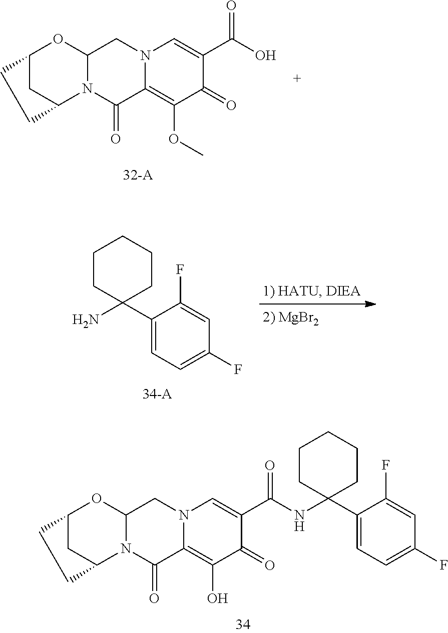 [SMILES: COc1c2n(cc(C(=O)O)c1=O)CC1O[C@H]3CC[C@H](C3)N1C2=O ; NC1(c2ccc(F)cc2F)CCCCC1 ; O=C(NC1(c2ccc(F)cc2F)CCCCC1)c1cn2c(c(O)c1=O)C(=O)N1C(C2)O[C@H]2CC[C@@H]1C2]

Compound 34 (32.8 mg, 82%) was obtained from compound 32-A (20.8 mg, 0.065 mmol) and compound 34-A (20.5 mg, 0.097 mmol) as described in the synthesis of compound 32. ¹H NMR (400 MHz, CDCl₃) δ 10.83 (s, 1H), ˜9.6 (br, 1H), 8.44 (s, 1H), 7.37 (td, J=9.0, 6.4 Hz, 1H), 6.97-6.76 (m, 1H), 6.69 (ddd, J=11.9, 8.8, 2.7 Hz, 1H), 5.48-5.18 (m, 2H), 4.68 (t, J=3.0 Hz, 1H), 4.28 (dd, J=13.1, 4.1 Hz, 1H), 4.03 (dd, J=13.0, 9.4 Hz, 1H), 2.60 (d, J=13.1 Hz, 2H), 2.29-1.96 (m, 4H), 1.95-1.77 (m, 4H), 1.77-1.65 (m, 4H), 1.61 (ddd, J=12.5, 4.1, 2.7 Hz, 1H), 1.30 (br, 1H). ¹⁹F NMR (376.1 MHz, CD₃CN) δ −76.41 (s, 3F), −107.86 (q, J=9.4 Hz, 1F), −113.13 (p, J=8.0 Hz, 1F). LCMS-ESI⁺ (m/z): [M+H]⁺ calculated for C₂₆H₂₈F₂N₃O₅: 500.20. found: 500.0.

### Example 35

### Preparation of Compound 35

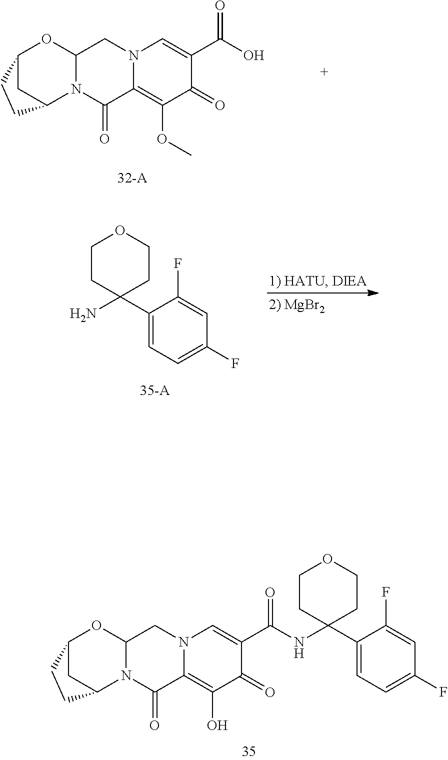 [SMILES: COc1c2n(cc(C(=O)O)c1=O)CC1O[C@H]3CC[C@H](C3)N1C2=O ; NC1(c2ccc(F)cc2F)CCOCC1 ; O=C(NC1(c2ccc(F)cc2F)CCOCC1)c1cn2c(c(O)c1=O)C(=O)N1C(C2)O[C@H]2CC[C@@H]1C2]

Compound 35 (30.4 mg, 76%) was obtained from compound 32-A (20.2 mg, 0.063 mmol) and compound 35-A (24.1 mg, 0.113 mmol) as described in the synthesis of compound 32. ¹H NMR (400 MHz, CDCl₃) δ 10.95 (s, 1H), 8.33 (s, 1H), ˜7.6 (br, 1H), 7.38 (td, J=9.0, 6.3 Hz, 1H), 6.85 (td, J=8.4, 2.6 Hz, 1H), 6.73 (ddd, J=11.7, 8.6, 2.6 Hz, 1H), 5.32 (dt, J=14.4, 4.0 Hz, 2H), 4.68 (t, J=3.1 Hz, 1H), 4.24 (dd, J=13.0, 3.9 Hz, 1H), 4.11-3.81 (m, 5H), 2.60 (d, J=13.7 Hz, 2H), 2.33-2.17 (m, 2H), 2.18-1.97 (m, 4H), 1.87 (m, 1H), 1.61 (dt, J=12.5, 3.3 Hz, 1H). ¹⁹F NMR (376.1 MHz, CD₃CN) δ −76.40 (s, 3F), −108.78 (q, J=10.3, 9.8 Hz, 1F), −112.63 (p, J=8.0 Hz, 1F). LCMS-ESI⁺ (m/z): [M+H]⁺ calculated for C₂₅H₂₆F₂N₃O₆: 502.18. found: 502.0.

### Example 36

### Preparation of Compound 36

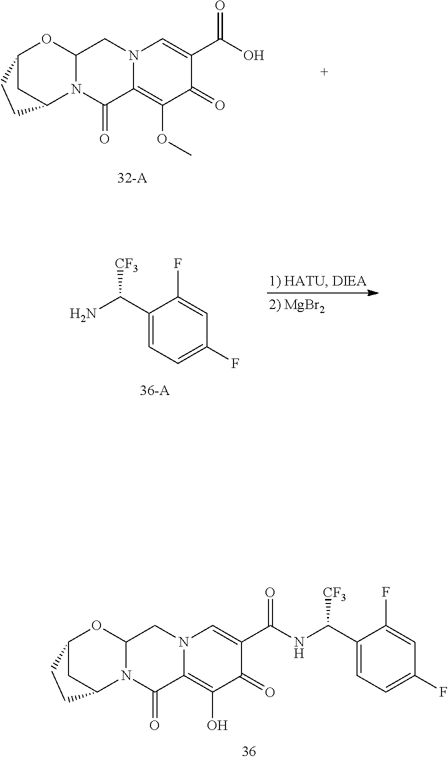 [SMILES: COc1c2n(cc(C(=O)O)c1=O)CC1O[C@H]3CC[C@H](C3)N1C2=O ; N[C@@H](c1ccc(F)cc1F)C(F)(F)F ; O=C(N[C@@H](c1ccc(F)cc1F)C(F)(F)F)c1cn2c(c(O)c1=O)C(=O)N1C(C2)O[C@H]2CC[C@@H]1C2]

Compound 36 (26 mg, 82%) was obtained from compound 32-A (20 mg, 0.062 mmol) and compound 36-A (22 mg, 0.089 mmol) as described in the synthesis of compound 32. ¹H NMR (400 MHz, CDCl₃) δ 11.31 (d, J=9.4 Hz, 1H), 8.41 (s, 1H), 7.65-7.44 (m, 1H), 6.95 (ddd, J=9.6, 5.6, 2.0 Hz, 1H), 6.92-6.79 (m, 1H), 6.15 (h, J=7.4 Hz, 1H), ˜6 (br, 1H), 5.41 (dd, J=9.5, 4.0 Hz, 1H), 5.31 (t, J=4.0 Hz, 1H), 4.70 (s, 1H), 4.34 (dd, J=12.8, 3.9 Hz, 1H), 4.05 (dd, J=12.9, 9.4 Hz, 1H), 2.26-1.99 (m, 4H), 1.99-1.87 (m, 1H), 1.62 (dt, J=12.6, 3.4 Hz, 1H). ¹⁹F NMR (376.1 MHz, CDCl₃) δ −75.23 (t, J=6.9 Hz, 3F), −76.33 (s, 3F), −108.31 (m, 1F), −112.30 (p, J=8.0 Hz, 1F). LCMS-ESI⁺ (m/z): [M+H]⁺ calculated for C₂₂H₁₉F₅N₃O₅: 500.12. found: 500.1.

### Example 37

### Preparation of Compound 37

### (3S,11aR)—N-(1-(2,4-difluorophenyl)cyclopropyl)-6-hydroxy-3-methyl-5,7-dioxo-2,3,5,7,11,11a-hexahydrooxazolo[3,2-a]pyrido[1,2-d]pyrazine-8-carboxamide

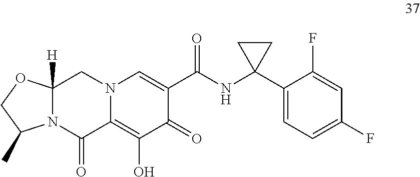 [SMILES: C[C@H]1CO[C@@H]2Cn3cc(C(=O)NC4(c5ccc(F)cc5F)CC4)c(=O)c(O)c3C(=O)N12]

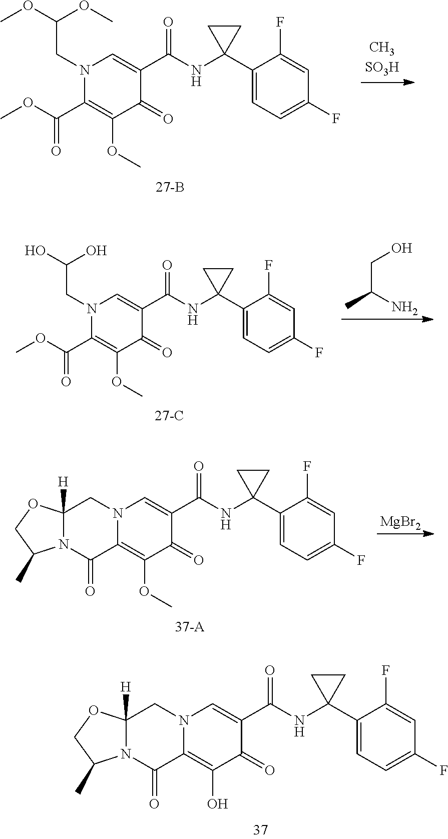

### Step 1

Methyl 5-(1-(2,4-difluorophenyl)cyclopropylcarbamoyl)-1-(2,2-dimethoxyethyl)-3-methoxy-4-oxo-1,4-dihydropyridine-2-carboxylate (27-B, 0.150 g, 0.32 mmol) in acetonitrile (1.5 mL) and acetic acid (0.2 mL) was treated with methanesulfonic acid (0.05 mL), sealed with a yellow cap, and heated to 70° C. After 16 hours, the mixture was cooled to afford a crude solution of methyl 5-(1-(2,4-difluorophenyl)cyclopropylcarbamoyl)-1-(2,2-dihydroxyethyl)-3-methoxy-4-oxo-1,4-dihydropyridine-2-carboxylate 27-C. LCMS-ESI⁺ (m/z): [M+H]⁺ calculated for C₁₈H₁₉F₂N₂O₇: 439. found: 439.

### Steps 2 and 3

Methyl 5-(1-(2,4-difluorophenyl)cyclopropylcarbamoyl)-1-(2,2-dihydroxyethyl)-3-methoxy-4-oxo-1,4-dihydropyridine-2-carboxylate (27-C, 0.32 mmol, the crude mixture from the previous step) was dissolved in acetonitrile (1.5 mL) and acetic acid (0.2 mL). (S)-2-aminopropan-1-ol (0.048 g, 0.64 mmol) and K₂CO₃ (0.088 g, 0.64 mmol) were added to the reaction mixture. The reaction mixture was sealed and heated to 70° C. After 3 hours, the reaction mixture was cooled and magnesium bromide (0.081 g, 0.44 mmol) was added. The mixture was resealed and heated to 50° C. After 10 minutes, the reaction mixture was cooled to 0° C. and 1 N hydrochloric acid (0.5 mL) was added in. Then the reaction mixture was diluted with MeOH (2 mL). After filtration, the crude was purified by Pre-HPLC purification (30-70% acetonitrile:water, 0.1% TFA) afforded Compound 37 (110 mg, 63%) as TFA salt. ¹H NMR (400 MHz, Methanol-d₄) δ 8.31 (s, 1H), 7.62 (td, J=9.2, 8.7, 6.5 Hz, 1H), 7.02-6.78 (m, 2H), 5.53-5.20 (m, 1H), 4.68 (dd, J=12.3, 4.2 Hz, 1H), 4.40 (dq, J=19.1, 6.7 Hz, 2H), 3.98 (dd, J=12.2, 10.0 Hz, 1H), 3.71 (dd, J=8.3, 6.3 Hz, 1H), 1.41 (d, J=6.1 Hz, 3H), 1.22 (s, 4H). ¹⁹F NMR (376 MHz, Methanol-d₄) δ −113.66-−113.95 (m, 1F), −113.94-−114.29 (m, 1F). LCMS-ESI⁺ (m/z): [M+H]⁺ calculated for C₂₁H₂₀F₂N₃O₅:432. found: 432.

### Antiviral Assay

### Example 38

### Antiviral Assays in MT4 Cells

For the antiviral assay utilizing MT4 cells, 0.4 μL of 189X test concentration of 3-fold serially diluted compound in DMSO was added to 40 μL of cell growth medium (RPMI 1640, 10% FBS, 1% penicilline/Streptomycine, 1% L-Glutamine, 1% HEPES) in each well of 384-well assay plates (10 concentrations) in quidruplicate.

1 mL aliquots of 2×10⁶ MT4 cells are pre-infected for 1 and 3 hours respectively at 37° C. with 25 μL (MT4) or of either cell growth medium (mock-infected) or a fresh 1:250 dilution of an HIV-IIIb concentrated ABI stock (0.004 m.o.i. for MT4 cells). Infected and uninfected cells are diluted in cell growth medium and 35 L of 2000 (for MT4) cells is added to each well of the assay plates.

Assay plates were then incubated in a 37° C. incubator. After 5 days of incubation, 25 μL of 2× concentrated CellTiter-Glo™ Reagent (catalog #G7573, Promega Biosciences, Inc., Madison, Wis.) was added to each well of the assay plate. Cell lysis was carried out by incubating at room temperature for 2-3 minutes, and then chemiluminescence was read using the Envision reader (PerkinElmer).

Compounds of the present invention demonstrate antiviral activity in this assay as depicted in Table 1 below. Accordingly, the compounds of the invention may be useful for treating the proliferation of the HIV virus, treating AIDS, or delaying the onset of AIDS or ARC symptoms.

TABLE 1

nM in MT-4

| Compound Number | EC₅₀ | CC₅₀ |
|---|---|---|
| 1 | 2.6 | 5819 |
| 2 | 1.9 | 2959 |
| 3 | 1.9 | 36185 |
| 4 | 14.8 | 45769 |
| 5 | 8.1 | 10452 |
| 6 | 5.3 | 53191 |
| 7 | 3.5 | 15610 |
| 8 | 2.5 | 13948 |
| 9 | 5.1 | 13451 |
| 10 | 6.1 | 3670 |
| 11 | 4.4 | 10249 |
| 12 | 5.4 | 3229 |
| 13 | 46.0 | 12666 |
| 14 | 65.5 | 4939 |
| 15 | 2.2 | 16268 |
| 16 | 1.5 | 13633 |
| 17 | 5.9 | 6613 |
| 18 | 4.1 | 10263 |
| 19 | 2.8 | 38690 |
| 20 | 3.3 | 27990 |
| 21 | 38.3 | 13010 |
| 22 | 64.3 | 4433 |
| 23 | 1.8 | 11528 |
| 24 | 3.4 | 12570 |
| 25 | 17.9 | 7066 |
| 26 | 8.0 | 11508 |
| 27 | 4.0 | 6828 |
| 28 | 15.6 | 18687 |
| 29 | 13.9 | 9446 |
| 30 | 4.4 | 8751 |
| 31 | 9.0 | 4525 |
| 32 | 14.0 | 4684 |
| 33 | 43.5 | 3971 |
| 34 | 455.9 | 3585 |
| 35 | 157.0 | 15546 |
| 36 | 3.5 | 13540 |
| 37 | 10 | 19486 |

### Example 39

### Human PXR Activation Assay

Luciferase Reporter Gene Assay.

A stably transformed tumor cell line (DPX2) plated on 96-well microtiter plates. DPX2 cells harbor the human PXR gene (NR1I2) and a luciferase reporter gene linked to two promoters identified in the human CYP3A4 gene, namely XREM and PXRE. The cells are treated with six concentrations of compounds (0.15˜50 μM) and incubated for 24 hr. The number of viable cells will be determined and the reporter gene activity is assessed. Positive control: Rifampicin at 6 concentrations (0.1˜20 □M). % Eₘₐₓ relative to the maximum fold induction by 10 or 20 □M RIF is calculated for test compounds according to the following equation which adjusts for the DMSO background: % Eₘₐₓ=(Fold induction−1)/(Maximum fold induction by RIF−1)×100%.

TABLE 2

| Compound Number | % Eₘₐₓ at 15 mM |
|---|---|
| 2 | 2.8 |
| 3 | 5.0 |
| 4 | 3.2 |
| 5 | 32 |
| 6 | 0.0 |
| 7 | 6.5 |
| 8 | 6.6 |
| 9 | 0.07 |
| 10 | 0.19 |
| 15 | 20 |
| 16 | 17 |
| 17 | 7.0 |
| 18 | 4.4 |
| 19 | 1.5 |
| 20 | 2.4 |
| 28 | 6.1 |
| 29 | 3.2 |
| 32 | 14 |
| 33 | 17 |
| 37 | 1.5 |

The data in Table 1 and 2 represent an average over time of each assays for each compound. For certain compounds, multiple assays have been conducted over the life of the project. Thus, the data reported in Tables 1 and 2 include the data reported in the priority document, as well as data from assays run in the intervening period.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A compound having the following Formula (I): 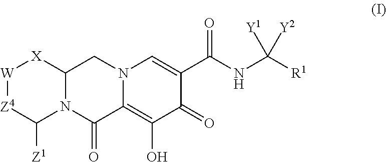
or a stereoisomer or pharmaceutically acceptable salt thereof,
wherein:
X is —O— or —NZ³— or —CHZ³—;
W is —O— or —NZ²— or —CHZ²—;
Z¹, Z² and Z³ are each, independently, hydrogen or C₁₋₃alkyl, or wherein Z¹ and Z² or Z¹ and Z³, taken together, form -L- wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—;
Z⁴ is a bond or —CH₂—, —CH₂CH₂—, —CH₂CH₂CH₂—, —CH₂OCH₂—, —CH₂NR^{a}CH₂—, —CH₂SCH₂—, —CH₂S(O)CH₂— or —CH₂SO₂CH₂—;
Y¹ and Y² are each, independently, hydrogen, C₁₋₃alkyl or C₁₋₃haloalkyl, or Y¹ and Y², together with the carbon atom to which they are attached, form a carbocyclic ring having from 3 to 6 ring atoms or a heterocyclic ring having from 3 to 6 ring atoms, wherein the carbocyclic or heterocyclic ring is optionally substituted with one or more R^{a};
R¹ is optionally substituted aryl or optionally substituted heteroaryl; and
each R^{a} is, independently, hydrogen, halo, hydroxyl or C₁₋₄alkyl, or wherein two R^{a} groups, together with the carbon atom to which they are attached, form C═O, and
wherein at least one of: (i) Z¹ and Z² or Z¹ and Z³, taken together, form -L-; or (ii) Y¹ and Y², together with the carbon atom to which they are attached, form a carbocyclic ring having from 3 to 5 ring atoms or a heterocyclic ring having from 3 to 5 ring atoms.

2. A compound of claim 1 wherein Z¹ and Z² or Z¹ and Z³, taken together, form -L-.

3. A compound of claim 2 having one of the following Formulas (II-A) or (II-B): 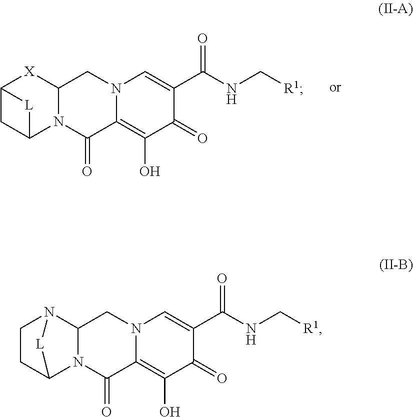
wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R^{a})₂SO₂NR^{a}C(R^{a})₂— or —C(R^{a})₂NR^{a}SO₂C(R^{a})₂—.

4. A compound of claim 1 wherein Y¹ and Y², together with the carbon atom to which they are attached, form a carbocyclic ring having from 3 to 5 ring atoms or a heterocyclic ring having from 3 to 5 ring atoms.

5. A compound of claim 4 having one of the following Formulas (III-A), (III-B), (III-C) or (III-D): 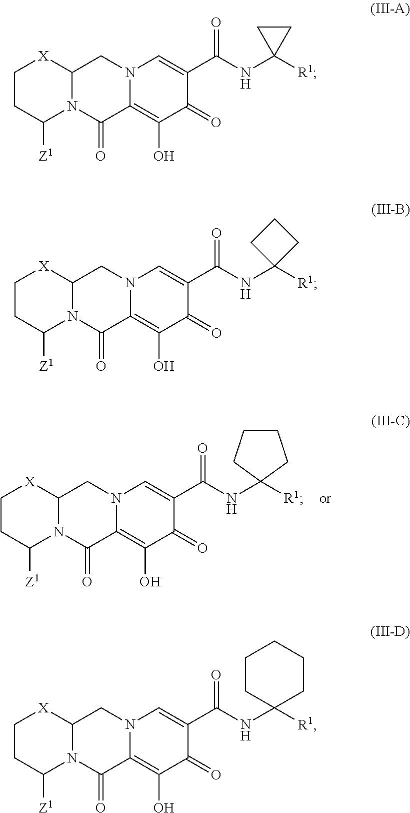
wherein Z¹ and Z³ are each, independently, hydrogen or C₁₋₃alkyl.

6. A compound of claim 4 having one of the following Formulas (III-E), (III-F), (III-G) or (III-H): 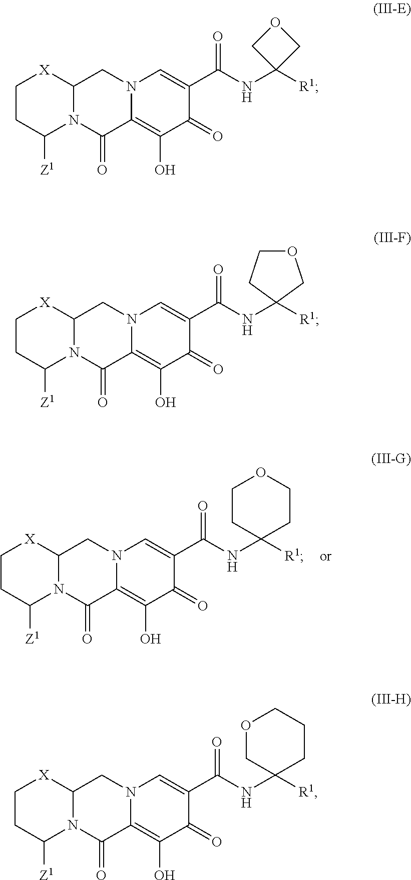
wherein Z¹ and Z³ are each, independently, hydrogen or C₁₋₃alkyl.

7. A compound of claim 1 wherein both (i) Z¹ and Z² or Z¹ and Z³, taken together, form -L-, and (ii) Y¹ and Y², together with the carbon atom to which they are attached, form a carbocyclic ring having from 3 to 5 ring atoms or a heterocyclic ring having from 3 to 5 ring atoms.

8. A compound of claim 7 having one of the following Formulas (IV-AA), (IV-AB), (IV-AC), (IV-AD), (IV-AE), (IV-AF), (IV-AG) or (IV-AH): 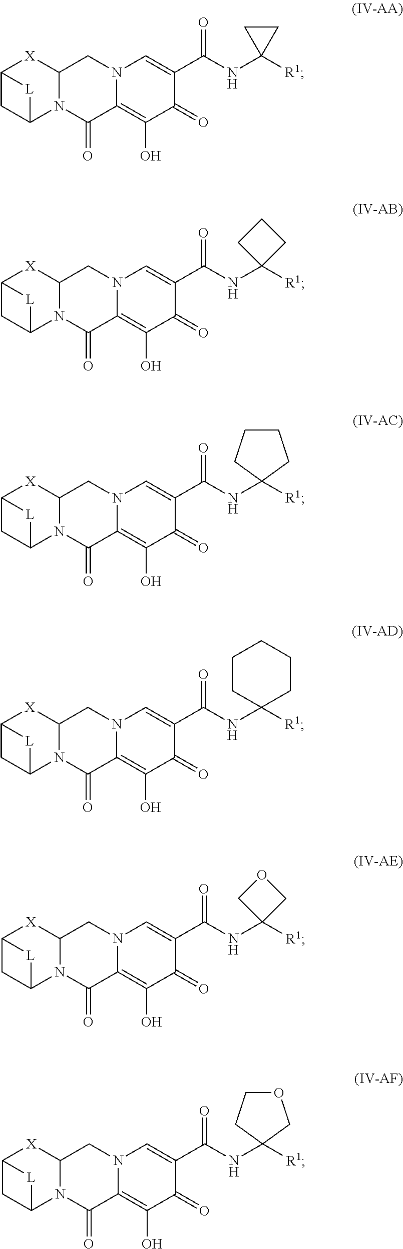 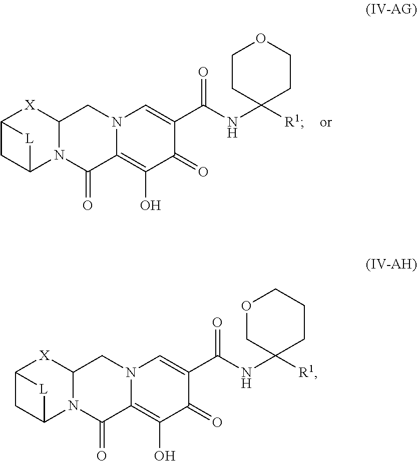
wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R)₂—, —C(R^{a})₂S(O)C(R^{a})₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R)₂SO₂NR^{a}C(R)₂— or —C(R^{a})₂NR^{a}SO₂C(R^{a})₂—.

9. A compound of claim 7 having one of the following Formulas (IV-BA), (IV-BB), (IV-BC), (IV-BD), (IV-BE), (IV-BF), (IV-BG) or (IV-BH): 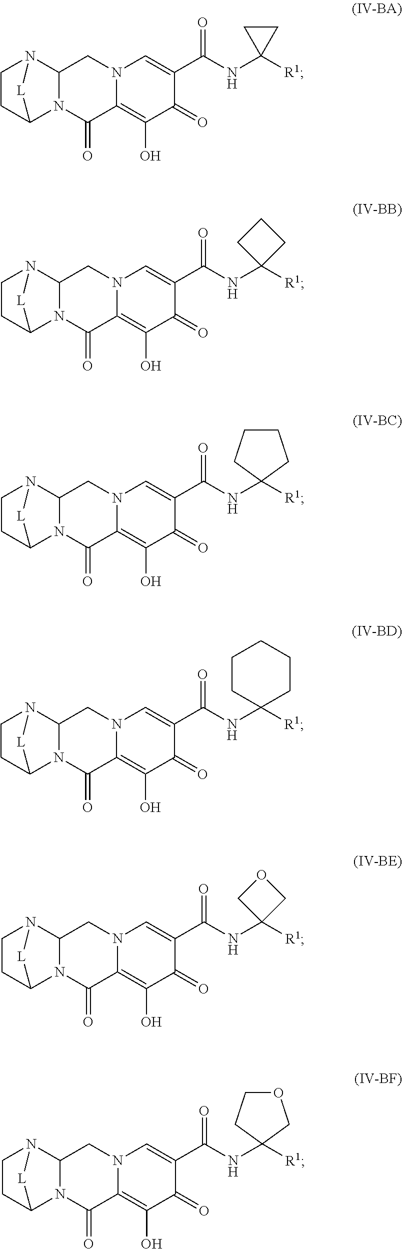 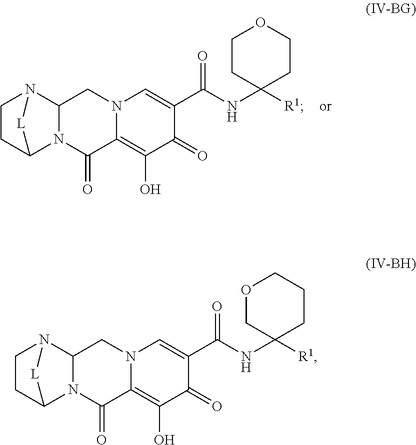
wherein L is —C(R^{a})₂—, —C(R^{a})₂C(R^{a})₂—, —C(R^{a})₂C(R^{a})₂C(R^{a})₂—, —C(R^{a})₂C(R^{a})₂C(R^{a})₂C(R^{a})₂—, —C(R^{a})₂OC(R^{a})₂—, —C(R^{a})₂NR^{a}C(R^{a})₂—, —C(R^{a})₂SC(R^{a})₂—, —C(R′)₂S(O)C(R′)₂—, —C(R^{a})₂SO₂C(R^{a})₂—, —C(R^{a})₂OC(R^{a})₂C(R^{a})₂—, —C(R^{a})₂C(R^{a})₂OC(R^{a})₂—, —C(R^{a})₂NR^{a}C(R^{a})₂C(R^{a})₂—, —C(R^{a})₂C(R^{a})₂NR^{a}C(R^{a})₂—, —C(R^{a})₂SC(R^{a})₂C(R^{a})₂—, —C(R^{a})₂C(R^{a})₂SC(R^{a})₂—, —C(R^{a})₂S(O)C(R^{a})₂C(R^{a})₂—, —C(R^{a})₂C(R^{a})₂S(O)C(R^{a})₂—, —C(R^{a})₂SO₂C(R^{a})₂C(R^{a})₂—, —C(R^{a})₂C(R^{a})₂SO₂C(R^{a})₂—, —C(R)₂SO₂NR^{a}C(R)₂— or —C(R^{a})₂NR^{a}SO₂C(R^{a})₂—.

10. A compound of any one of claims 1-3 or 7-9 wherein L is —C(R^{a})₂—, —C(R^{a})₂C(R^{a})₂—, —C(R^{a})₂C(R^{a})₂C(R^{a})₂—, or —C(R^{a})₂C(R^{a})₂C(R^{a})₂C(R^{a})₂—.

11. A compound of claim 10 wherein L is —C(R^{a})₂—.

12. A compound of claim 10 wherein L is —C(R^{a})₂C(R^{a})₂—.

13. A compound of claim 10 wherein L is —C(R^{a})₂C(R^{a})₂C(R^{a})₂—.

14. A compound of any one of claims 1-3 or 7-13 wherein each R^{a} is hydrogen.

15. A compound of any one of claims 1-3 or 7-9 wherein L is —C(R^{a})₂OC(R^{a})₂—, —C(R^{a})₂NR^{a}C(R^{a})₂—, —C(R^{a})₂SC(R^{a})₂—, —C(R^{a})₂S(O)C(R^{a})₂—, or —C(R^{a})₂SO₂C(R^{a})₂—.

16. A compound of claim 15 wherein each R^{a} is hydrogen.

17. A compound of any one of claims 1-8 or 10-16 wherein X is —O—.

18. A compound of any one of claims 1-8 or 10-16 wherein X is —NH—.

19. A compound of any one of claims 1-8 or 10-16 wherein X is —CH₂—.

20. A compound of any one of claims 1-19 wherein R¹ is aryl substituted with at least one halogen.

21. A compound of any one of claims 1-20 wherein R¹ is aryl substituted with one or two halogens.

22. A compound of claim 20 wherein R¹ is 2,4-difluorophenyl, 4-fluorophenyl, 2,3-difluorophenyl, 3-fluoro-4-chlorophenyl, 3,4-difluorophenyl, 2-fluoro-4-chlorophenyl, 2-fluorophenyl, 3,5-difluorophenyl or 3-trifluoromethyl-4-fluorophenyl.

23. A compound of claim 22 wherein R¹ is 2,4-difluorophenyl.

24. A pharmaceutical composition comprising a compound of any one of claims 1-23, or a stereoisomer or pharmaceutically acceptable salt thereof, and a pharmaceutically acceptable carrier, diluent or excipient.

25. A method of treating or preventing an HIV infection in a human having or at risk of having the infection by administering to the human a therapeutically effective amount of a compound of any one of claims 1-23 or a pharmaceutical composition of claim 24.

26. Use of a compound of any one of claims 1-23 or a pharmaceutical composition of claim 24 for the treatment or prevention of an HIV infection in a human having or at risk of having the infection.

27. A compound as described in any one of claims 1-23, or a pharmaceutically acceptable salt thereof for use in medical therapy.

28. A compound as described in any one of claims 1-23, or a pharmaceutically acceptable salt thereof, for use in the prophylactic or therapeutic treatment of an HIV infection.
